(19) United States
(12) Patent Application Publication
(10) Pub. No.: US 2024/0269129 A1
(43) Pub. Date: Aug. 15, 2024

(54) GLYCOGEN SYNTHASE KINASE 3 (GSK3) INHIBITORS FOR TREATING CTNNB1 SYNDROME

(71) Applicants: Trustees of Tufts College, Medford, MA (US); The Broad Institute, Inc., Cambridge, MA (US)

(72) Inventors: Michele Jacob, Medford, MA (US); Jonathan Alexander, Medford, MA (US); Florence Wagner, Cambridge, MA (US); Michel Weiwer, Cambridge, MA (US)

(21) Appl. No.: 18/622,123

(22) Filed: Mar. 29, 2024

Related U.S. Application Data
(63) Continuation-in-part of application No. PCT/US2022/077411, filed on Sep. 30, 2022.
(60) Provisional application No. 63/261,919, filed on Sep. 30, 2021.

(51) Int. Cl. A61K 31/4745; A61P 25/28

(52) U.S. Cl. CPC A61K 31/4745; A61P 25/28

(57) ABSTRACT

Disclosed are methods and compositions for treating CTNNB1 syndrome. The disclosed methods and compositions may utilize or comprise a glycogen synthase kinase 3 (GSK3) inhibitor, or a pharmaceutically acceptable salt thereof. The GSK3 inhibitor may comprise small molecules such as substituted tricyclic pyrazolo-tetrahydroquinolinones, and the GSK3 inhibitor may selectively inhibit GSK3β and GSK3α or dually inhibit GSK3α and GSK3β.

### CROSS-REFERENCE TO RELATED APPLICATIONS

This application is a continuation-in-part of and claims the benefit of PCT/US2022/077411, filed Sep. 30, 2022, which is based on and claims the benefit of U.S. Provisional Patent Application No. 63/261,919, filed Sep. 30, 2021, each of which is incorporated by reference herein in their entireties.

### STATEMENT REGARDING FEDERALLY SPONSORED RESEARCH OR DEVELOPMENT

This invention was made with government support under Grant No. NS119958 awarded by the National Institutes of Health. The government has certain rights in this invention.

### BACKGROUND

The field of the invention relates to glycogen synthase kinase 3 (GSK3) inhibitors that are useful for treatment of CTNNB1 syndrome. In particular, the field of the invention relates to small molecules that are inhibitors of the glycogen synthase kinase 3 (GSK3), and the use of such compounds in pharmaceutical compositions for treating diseases and/or disorders associated with CTNNB1 syndrome.

CTNNB1 syndrome is a human developmental disorder characterized by intellectual disabilities, microcephaly, motor and speech delays, truncal hypotonia, peripheral hypertonia, spasticity, and visual defects (usually mild). It is caused by CTNNB1 (β-catenin) haploinsufficiency due to partial or complete deletion mutations. CTNNB1 is a high-confidence risk gene for intellectual disabilities. Treatments for CTNNB1 Syndrome are lacking due to limited knowledge of the underlying pathophysiological changes and limited studies of in vivo mouse and in vitro human cell models of CTNNB1 haploinsufficiency.

CTNNB1 Syndrome is a rare disease-impacting an estimated 1 in 50,000. To date, over 300 children with CTNNB1 Syndrome have been definitively diagnosed by whole exome sequencing. CTNNB1 mutations have also been identified recently in children diagnosed with Rett syndrome and cerebral palsy. The numbers are increasing as more children with relevant symptoms undergo genetic testing. Several other human gene mutations also cause reduced β-catenin levels or functions and similar developmental disorders, intellectual disabilities and autism spectrum disorders. These disorders may share a common pathology and benefit from the therapeutic strategies that are identified for CTNNB1 syndrome. So far, there are no treatments for CTNNB1 syndrome.

Previously, studies have shown that the small molecule GSK3 inhibitors display unparalleled selectivity, brain bioavailability and favorable pharmacokinetic (PK)/pharmacodynamic (PD) profiles in in vivo mouse studies, including sustained brain and peripheral exposures, assessed up to 8 hours, after a single intraperitoneal (IP) dose (30 mg/kg), with a brain/plasma ratio of 0.16 (Wagner et al., 2016, 2018). These new, next generation GSK3α,β inhibitors overcome limitations seen in clinical and preclinical studies of other current GSK3 inhibitors due to sub-optimal potency or brain exposure and less kinome selectivity when evaluated in large-scale screens, supporting their potential for the best efficacy and safety outcomes (Bernard-Gauthier et al., 2019). Previous dose-response studies of GSK3α,β inhibitor BRD0320 show that 10 μM concentration increases β-catenin in mouse and human non-neuronal cell lines (Wagner et al., 2018).

As such, treatment methods for diseases and disorders associated with CTNNB1 syndrome are desirable. In particular, drug treatments that correct β-catenin levels and associated molecular changes need to be identified for advancing the future design of therapeutic strategies for patients with CTNNB1 syndrome.

### SUMMARY

Disclosed are methods and pharmaceutical compositions for treating CTNNB1 syndrome. The disclosed methods utilize and the pharmaceutical compositions comprise one or more GSK3 inhibitors, e.g., GSK3α inhibitors, GSK3β inhibitors, and dual inhibitors of GSK3a and GSK3β, that modulate β-catenin levels and associated molecular changes in the subject in need thereof, thereby treating CTNNB1 syndrome.

The disclosed methods and compositions may include a small molecule GSK3 inhibitor comprising a substituted tricyclic pyrazolo-tetrahydroquinolinone. In some embodiments, the GSK3 inhibitor dually inhibits both GSK3α and GSK3β paralogs, rather than selectively inhibiting GSK3β or selectively inhibiting GSK3a.

In some embodiments, the disclosed methods for treating CTNNB1 syndrome may comprise administering to a subject in need thereof an effective amount of one or more GSK3 inhibitors, or a pharmaceutically acceptable salt thereof, or a pharmaceutical composition comprising an effective amount of the one or more GSK3 inhibitors, or a pharmaceutically acceptable salt thereof, together with a pharmaceutical excipient, carrier, or diluent. In some embodiments, the GSK3 inhibitor dually inhibits both GSK3α and GSK3β paralogs, rather than selectively inhibiting GSK3β or selectively inhibiting GSK3α.

The GSK3 inhibitors may have a Formula I:

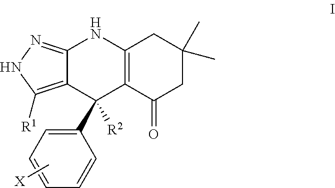

wherein X is hydrogen or halogen;
R¹ is alkyl, unsubstituted or substituted cycloalkyl optionally substituted at one or more positions with halogen, or halogen; and
R² is alkyl.

The disclosed compounds for treating CTNNB1 syndrome may be used to prepare and formulate pharmaceutical compositions. As such, also disclosed herein are pharmaceutical compositions comprising an effective amount of any of the compounds disclosed herein, or pharmaceutically acceptable salts of any of the compounds disclosed herein, together with a pharmaceutically acceptable excipient, carrier, or diluent.

In some embodiments, the disclosed GSK3 inhibitors may be used for preparing a medicament for treating a disease or disorder associated with CTNNB1 syndrome, and in particular, a disease or disorder associated with CTNNB1 syndrome that may be treated with an inhibitor of GSK3. In some embodiments, the disclosed GSK3 inhibitors may exhibit greater specificity for GSK3β or GSK3a. In other embodiments, the disclosed GSK3 inhibitors may dually inhibit both GSK3α and GSK3β paralogs.

### BRIEF DESCRIPTION OF THE DRAWINGS

FIG. 1. The β-catenin heterozygous mice (BcatHet) display 50% decreases in β-catenin protein and mRNA levels, relative to littermate controls. Muscle, brain, and monocytes were tested for GSKα and GSKβ.

FIG. 2. The β-catenin heterozygous mice exhibit reduced associative learning, relative to littermate controls. Classic spatial learning task that pairs association of a particular context with a mild foot shock; freezing in the context is measured as an indicator of learning; n=26 control, 24 BcatHet; p<0.0001 Repeated Measure ANOVA; **p<0.01, ***p<0.001 multiple comparison t-test.

FIG. 3. The β-catenin heterozygous mice exhibit reduced muscle grip strength, relative to littermate controls; n=9-10 per genotype; p=0.0029 (Average) and p=0.0106 (Max) Student t-test.

FIG. 4. The β-catenin heterozygous mice exhibit reduced motor learning, but no differences in velocity of movement (or distance travelled). n=9-10 per genotype; Rotarod: p=0.0003 RM ANOVA, *p<0.05,***p<0.001 multiple comparison t-test; Velocity in open field test, p=0.693, Student t-test.

FIG. 5. β-catenin protein levels in the young adult β-catenin heterozygous mouse brain and skeletal muscle are significantly increased by treatment with the dual GSK3α/β and GSK3β selective inhibitors (BRD0320 and BRD3731, respectively), but are not increased by the GSKα selective inhibitor (BRD0705), relative to vehicle treated β-catenin heterozygote mouse levels, as measured by quantitative immunoblotting. β-catenin protein levels in β-catenin heterozygous mice treated with the dual inhibitor resemble baseline wildtype littermate levels. n=12 Control, 12 Beat Het (Vehicle), 12 Beat Het (Dual inhibit), 6 (β inhibitor), 6-12 (α inhibitor); p<0.001 One-way ANOVA; *p<0.05, **p<0.01, ***p<0.001 multiple t-test correction.

FIG. 6. BRD0320, a GSK3α/β dual paralog inhibitor, treated young adult CTNNB1/β-catenin heterozygous mice show significant improvements in learning; n=13 Control, 11 Beat Het (Vehicle), 11 Beat Het; p<0.001 Repeated Measure ANOVA; *p<0.05 **p<0.01***p<0.001, p<0.0001 multiple t-test correction.

FIG. 7. BRD0320 treated young adult β-catenin heterozygous mice display significant improvements in muscle grip strength.

FIG. 8. BRD0705, a GSK3α selective inhibitor, displays a slight trend (not significant) toward improvements in learning and muscle grip strength; n=13 Control, Beat Het (Vehicle), 14 Beat Het (a selective inhibitor); *p<0.05 **p<0.01***p<0.001, multiple t-test correction.

FIG. 9. BRD3731, a GSK3β selective inhibitor, displays significant improvement in learning (trial 4 in the contextual conditional fear learning task) and a trend toward improvement in muscle grip strength; n=13 Control, 11 Beat Het (Vehicle), 11 Beat Het (P selective inhibitor); *p<0.05 **p<0.01***p<0.001, multiple t-test correction. p<0.001 Repeated Measure ANOVA; *p<0.05 **p<0.01***p<0.001, p<0.0001 multiple t-test correction

FIG. 10. BRD0320, a GSK3α/β dual paralog inhibitor does not alter (3-catenin protein levels in young adult wildtype littermate mice treated with the same paradigm that provided correction of β-catenin protein levels in the β-catenin heterozygous mice. N=4 Wildtype Control (Vehicle), 5 (Wildtype Control (dual inhibitor treated), Student's t-test

FIG. 11. BRD0320, a GSK3α/β dual paralog inhibitor caused no significant changes (no adverse effects) in learning and muscle grip strength of wildtype littermate mice. n=11 Control (Vehicle), 12 Control (dual inhibitor treated), Repeated Measure ANOVA p=0.89178

FIG. 12. Design and characterization of first paralog selective inhibitors of GSK3α and GSK3β. (a) General synthetic scheme for synthesis of the pyrazolo-tetrahydroquinolinone scaffold. (b) IC₅₀ GSK3α and GSK3β values for inhibition were determined at Km of ATP in a motility-shift microfluidic assay (Caliper, MA) measuring phosphorylation of a synthetic substrate. Values are average of three or more experiments. Data are shown as IC₅₀ values in μM±standard deviation. Compounds were tested in duplicate in a 12-point dose curve with 3-fold dilution starting at 33.3 μM. (c) Kinome-wide selectivity for BRD0705 and BRD3731 (GSK3β selective inhibitor) represented on a kinome phylogenetic tree. Each inhibitor was screened against 311 kinases at a 10 μM concentration. Kinases with >50% inhibition are depicted (percentage inhibition proportional to size of red dot).

FIG. 13. GSK3α and GSK3β selective inhibitors play a differential role on β-catenin stabilization in a context dependent manner. (a) Biophysical measure of GSK3 target engagement in HEK 293 cells by BRET signal between a NanoLuc fused protein target and a small molecule labeled with the NanoBRET acceptor dye. Kds for each inhibitor are reported as mean±SEM of two replicates. (b) β-catenin immunofluorescence staining in HEK 293 T after treatment with the indicated inhibitor β-catenin in red and DAPI in blue). (c) β-catenin TCF/LEF luciferase reporter assay in HEK 293T after treatment with the indicated inhibitor. * p-value<0.05 calculated using a Mann-Whitney test in comparison with control conditions. Error bars represent mean±SEM of ten replicates. (d-e) Western immunoblot for β-catenin and vinculin after treatment with the indicated inhibitors in HL-60. (f) Western immunoblot for β-catenin, p-β-catenin (S675), p-β-catenin (S33/37/T41) and actin after treatment with the indicated inhibitors in TF-1. (g) β-catenin TCF/LEF luciferase reporter assay in TF-1 after 24 hours of treatment with the indicated inhibitor. * p-value<0.05 is calculated using a Welch's t test in comparison with control conditions. Error bars represent mean±SEM of three replicates. (h-i) Western immunoblot for β-catenin and actin after treatment with the indicated inhibitors in MV4-11 (h) and MLL-AF9 murine leukemic cells (i).

FIG. 14. FIG. 1. GSK3α,β inhibitors increase β-cat protein levels in (a) human CTNNB1 heterozygous glutamatergic neurons treated with BRD0320, relative to vehicle-treated het neurons, and (b) CTNNB1 het iPSCs treated with BRD0320 and LY2090314 at the indicated concentrations. Wnt target, DKK1 protein levels show similar increases to β-cat. Histograms showing comparison to (a) WT neuron levels set as 1 and (b) vehicle-treated het iPSC levels set as 1. LY2090314 increases β-cat protein levels at 10× lower concentration relative to BRD0320.

### DETAILED DESCRIPTION

The present invention is described herein using several definitions, as set forth below and throughout the application.

Unless otherwise specified or indicated by context, the terms “a”, “an”, and “the” mean “one or more.” For example, “a substitution” should be interpreted to mean “one or more substitutions.” Similarly, “a substituent group” should be interpreted to mean “one or more substituent groups.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus<10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Disclosed are methods and pharmaceutical compositions for treating CTNNB1 syndrome. The disclosed methods utilize and the pharmaceutical compositions comprise one or more GSK3 inhibitors. The GSK3 inhibitor may be a GSK3α and GSK3β dual paralog inhibitors. GSK3β inhibitors may include small molecules such as substituted tricyclic pyrrazolo-tetrahydroquinolinone, disclosed in Wagner et al. 2018, Sci Transl Med. 10(431) and in U.S. Pat. No. 10,137,122, the contents of which are incorporated herein by reference in their entireties.

The disclosed GSK3 inhibitors may include a substituted tricyclic pyrrazolo-tetrahydroquinolinone. In some embodiments, the disclosed GSK3 inhibitors have a Formula I:

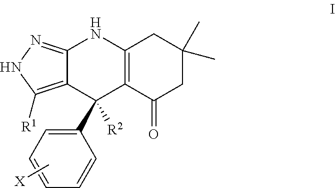

wherein X is hydrogen or halogen;
R¹ is alkyl, unsubstituted or substituted cycloalkyl optionally substituted at one or more positions with halogen, or halogen; and
R² is alkyl.

In some embodiments, the X in the compound of Formula I is hydrogen. In other embodiments, the X in the compound of Formula I is a halogen, e.g., F.

In some embodiments, the R¹ group is an unsubstituted or substituted cycloalkyl, e.g., cycloproyl or cyclobutyl, optionally substituted at one or more positions with halogen, e.g., F.

In some embodiments, the R¹ group is an alkyl, e.g., methyl or neopentyl.

In some embodiments, the R¹ group is a halogen, e.g., Cl.

In some embodiments, the R² group in the compound of Formula I is a methyl, ethyl group, or i-propyl.

In some embodiments, X is hydrogen, R¹ is chloro, and R² is methyl in compounds of Formula I.

In some embodiments, X is hydrogen, R¹ is cyclopropyl, and R² is methyl in compounds of Formula I.

In some embodiments, X is fluoro, R¹ is cyclopropyl, and R² is methyl in compounds of Formula I.

In some embodiments, X is hydrogen, R¹ is difluoro-substituted cyclobutyl, and R² is methyl in compounds of Formula I.

In some embodiments, X is hydrogen, R¹ is neopentyl, and R² is methyl in compounds of Formula I.

In some embodiments, X is hydrogen, R¹ is methyl, and R² is methyl in compounds of Formula I.

In some embodiments, X is hydrogen, R¹ is cyclobutyl, and R² is methyl in compounds of Formula I.

The disclosed GSK3 inhibitors may include those shown in Table. 1.

TABLE 1
GSK3 inhibitors.

| Compound | IUPAC name | Structure |
|---|---|---|
| LY2090314 | 3-(9-fluoro-2-(piperidine-1- carbonyl)-1,2,3,4-tetrahydro- [1,4]diazepino[6,7,1-hi]indol-7-yl)- 4-(imidazo[1,2-a]pyridin-3-yl)-1H- pyrrole-2,5-dione | 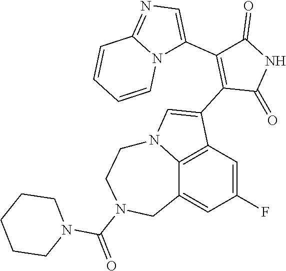 [SMILES: O=C1NC(=O)C(c2cnc3ccccn23)=C1c1cn2c3c(cc(F)cc13)CN(C(=O)N1CCCCC1)CC2] |
| 9-ING-41 | 3-(5-fluorobenzofuran-3-yl)-4-(5- methyl-5H-[1,3]dioxolo[4,5- f]indol-7-yl)-1H-pyrrole-2,5-dione | 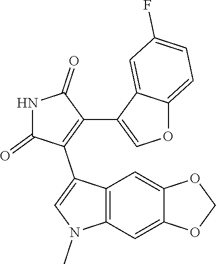 [SMILES: Cn1cc(C2=C(c3coc4ccc(F)cc34)C(=O)NC2=O)c2cc3c(cc21)OCO3] |
| AZD1080 | 2-hydroxy-3-(5- (morpholinomethyl)pyridin-2-yl)- 1H-indole-5-carbonitrile | 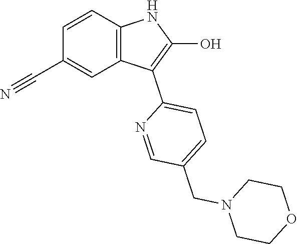 [SMILES: N#Cc1ccc2[nH]c(O)c(-c3ccc(CN4CCOCC4)cn3)c2c1] |
| AZD7969 | 3-amino-6-(4-(3-((3- methoxypropyl)amino)propyl)phen yl)-N-(pyridin-3-yl)pyrazine-2- carboxamide | 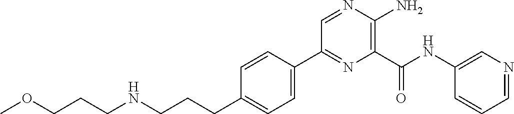 [SMILES: COCCCNCCCc1ccc(-c2cnc(N)c(C(=O)Nc3cccnc3)n2)cc1] |
| AZD2858 | 3-amino-6-(4-((4-methylpiperazin- 1-yl)sulfonyl)phenyl)-N-(pyridin-3- yl)pyrazine-2-carboxamide | 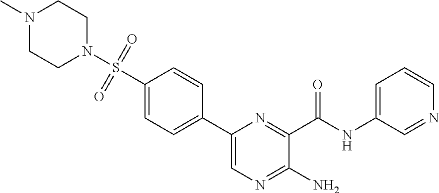 [SMILES: CN1CCN(S(=O)(=O)c2ccc(-c3cnc(N)c(C(=O)Nc4cccnc4)n3)cc2)CC1] |
| AZ13282107 | 5-fluoro-N-(3-fluoro-4- (methylsulfonyl)phenyl)-4-(1- (tetrahydro-2H-pyran-4-yl)-1H- imidazol-5-yl)pyrimidin-2-amine | 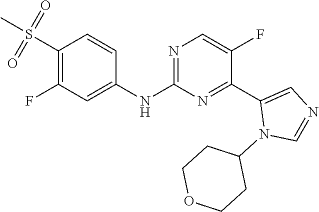 [SMILES: CS(=O)(=O)c1ccc(Nc2ncc(F)c(-c3cncn3C3CCOCC3)n2)cc1F] |
| BMS cpd33 | N-(4-(4-chlorophenyl)pyridin-3- yl)-2- (cyclopropanecarboxamido) isonicotinamide | 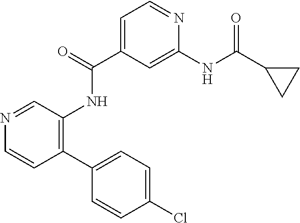 [SMILES: O=C(Nc1cnccc1-c1ccc(Cl)cc1)c1ccnc(NC(=O)C2CC2)c1] |
| BMS cpd34 | 2-((4-cyanophenyl)amino)-N-(4- (4-fluorophenyl)pyridin-3- yl)pyrimidine-4-carboxamide | 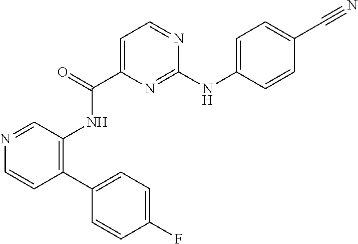 [SMILES: N#Cc1ccc(Nc2nccc(C(=O)Nc3cnccc3-c3ccc(F)cc3)n2)cc1] |
| BMS cpd 40 | 2-((4-fluorophenyl)amino)-N-(4- (4-fluorophenyl)pyridin-3- yl)pyrimidine-4-carboxamide | 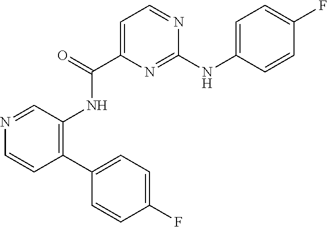 [SMILES: O=C(Nc1cnccc1-c1ccc(F)cc1)c1ccnc(Nc2ccc(F)cc2)n1] |
| BMS cpd 45 | 2-((4- (difluoromethoxy)phenyl)amino)- N-(4-(4-fluorophenyl)pyridin-3- yl)pyrimidine-4-carboxamide | 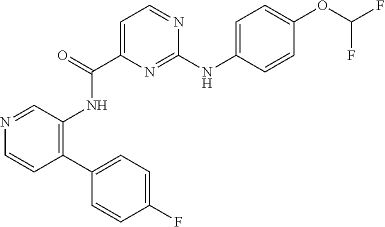 [SMILES: O=C(Nc1cnccc1-c1ccc(F)cc1)c1ccnc(Nc2ccc(OC(F)F)cc2)n1] |
| SAR502250 | (S)-2-(2-(4- fluorophenyl)morpholino)-1- methyl-[4,4′-bipyrimidin]-6(1H)- one | 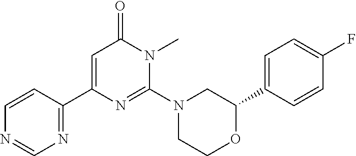 [SMILES: Cn1c(N2CCO[C@@H](c3ccc(F)cc3)C2)nc(-c2ccncn2)cc1=O] |
| Tideglusib | 4-benzyl-2-(naphthalen-1-yl)- 1,2,4-thiadiazolidine-3,5-dione | 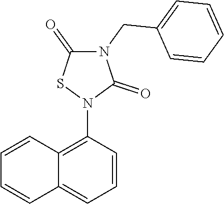 [SMILES: O=c1sn(-c2cccc3ccccc23)c(=O)n1Cc1ccccc1] |
| CHIR-98014 | N2-(2-((4-(2,4-dichlorophenyl)- 5-(1H-imidazol-1-yl)pyrimidin- 2-yl)amino)ethyl)-5- nitropyridine-2,6-diamine | 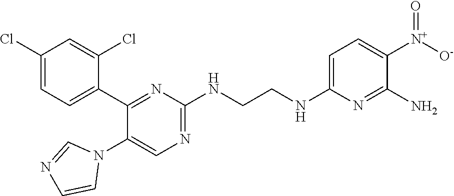 [SMILES: Nc1nc(NCCNc2ncc(-n3ccnc3)c(-c3ccc(Cl)cc3Cl)n2)ccc1[N+](=O)[O-]] |
| TDZD-8 | 4-benzyl-2-methyl-1,2,4- thiadiazolidine-3,5-dione | 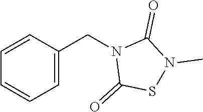 |
| AR-A014418 | 1-(4-methoxybenzyl)-3-(5- nitrothiazol-2-yl)urea | 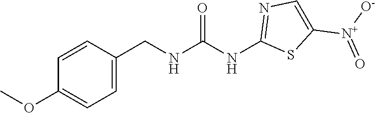 [SMILES: COc1ccc(CNC(=O)Nc2ncc([N+](=O)[O-])s2)cc1] |
| SB-415286 | 3-((3-chloro-4- hydroxyphenyl)amino)-4-(2- nitrophenyl)-1H-pyrrole-2,5- dione | 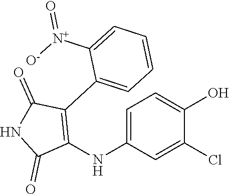 [SMILES: O=C1NC(=O)C(c2ccccc2[N+](=O)[O-])=C1Nc1ccc(O)c(Cl)c1] |
| SB216763 | 3-(2,4-dichlorophenyl)-4-(1- methyl-1H-indol-3-yl)-1H- pyrrole-2,5-dione | 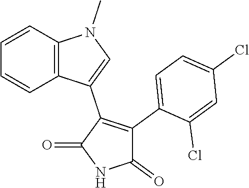 [SMILES: Cn1cc(C2=C(c3ccc(Cl)cc3Cl)C(=O)NC2=O)c2ccccc21] |
| TWS119 | 3-((6-(3-aminophenyl)-7H- pyrrolo[2,3-d]pyrimidin-4- yl)oxy)phenol | 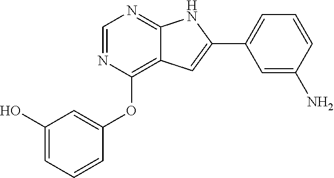 [SMILES: Nc1cccc(-c2cc3c(Oc4cccc(O)c4)ncnc3[nH]2)c1] |
| 1- Azakenpaullone | 9-bromo-7,12- dihydropyrido[3′,2′:2,3]azepino [4,5-b]indol-6(5H)-one | 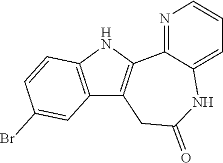 [SMILES: O=C1Cc2c([nH]c3ccc(Br)cc23)-c2ncccc2N1] |
| Kenpaullone | 9-bromo-7,12- dihydrobenzo[2,3]azepino[4,5- b]indol-6(5H)-one | 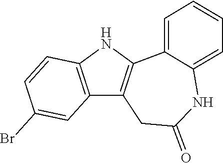 [SMILES: O=C1Cc2c([nH]c3ccc(Br)cc23)-c2ccccc2N1] |
| TCS 2002 | 2-methyl-5-(3-(4- (methylsulfinyl)phenyl) benzofuran-5-yl)-1,3,4- oxadiazole | 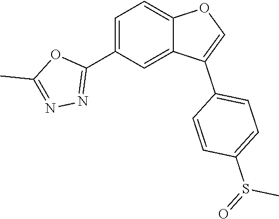 |
| PT-65 | 3-amino-6-(4-((4-(4-(1-(17-((2- (2,6-dioxopiperidin-3-yl)-1,3- dioxoisoindolin-4-yl)amino)- 3,6,9,12,15- pentaoxaheptadecyl)-1H-1,2,3- triazol-4-yl)butyl)piperazin-1- yl)sulfonyl)phenyl)-N-(pyridin- 3-yl)pyrazine-2-carboxamide | 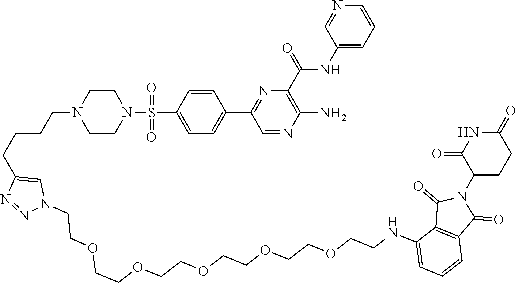 [SMILES: Nc1ncc(-c2ccc(S(=O)(=O)N3CCN(CCCCc4cn(CCOCCOCCOCCOCCOCCNc5cccc6c5C(=O)N(C5CCC(=O)NC5=O)C6=O)nn4)CC3)cc2)nc1C(=O)Nc1cccnc1] |

The GSK3 inhibitors may be optically pure with respect to one or more chiral centers (e.g., some or all of the chiral centers may be completely in the S configuration; and/or some or all of the chiral centers may be completely in the R configuration; etc.). Additionally or alternatively, one or more of the chiral centers may be present as a mixture of configurations (e.g., a racemic or another mixture of the R configuration and the S configuration). Compositions comprising substantially purified stereoisomers, epimers, or enantiomers of compounds described herein are contemplated herein (e.g., a composition comprising at least about 90%, 95%, or 99% pure stereoisomer, epimer, or enantiomer.)

As used herein, “CTNNB1 syndrome” refers to diseases or disorders caused by either a complete deletion, partial deletion or mutation of the CTNNB1 gene. The CTNNB1 gene provides instructions for making the protein beta-catenin, which is present in all cell types and tissues and is primarily found at junctions that connect neighboring cells (adherens junctions, synaptic complexes) and in the nucleus. CTNNB1 Syndrome symptoms range from mild developmental delays to severe physical and intellectual disabilities, including global developmental delay, spasticity, truncal hypotonia, peripheral hypertonia, microcephaly, amblyopia, strabismus, and hyperopia.

The GSK3 inhibitors disclosed herein may exhibit specificity for GSK3β and GSK3a. As used herein, “GSK3” refers to glycogen synthase kinase 3, which is a key regulatory kinase in the WNT pathway. As used herein, “GSK3β” refers to glycogen synthase kinase 3 beta, which is a multifunctional serine/threonine kinase and an enzyme that in humans is encoded by the GSK3β gene. As used herein, “GSK3a” refers to glycogen synthase kinase 3 alpha.

As used herein, the term “specificity for GSK3a” may be used to refer to GSK3 inhibitors that specifically or selectively inhibit GSK3a, relative to other glycogen synthase kinases, such as GSK3β. For example, a GSK3 inhibitor that specifically inhibits GSK3α may have an IC₅₀ (μM) that is lower than an IC₅₀ for another glycogen synthase kinase, such as GSK3β. A GSK3 inhibitor that specifically inhibits GSK3α over another glycogen synthase kinase, such as GSK3β, has an IC₅₀ (μM) for GSK3α that is at least 2-times lower, at least 3-times lower, at least 5-times lower, at least 10-times lower, at least 20-times lower, at least 50-times lower, at least 100-times lower, at least 500-times lower, or at least 1000-times lower, than the IC₅₀ (μM) for the other glycogen synthase kinase, such as GSK3β. In some embodiments, the GSK3 inhibitor has an IC₅₀ for GSK3α of less than about 0.050 μM, 0.040 μM, 0.030 μM, 0.020 μM, or 0.010 μM. In some embodiments, the GSK3 inhibitor has an IC₅₀ for GSK3β of greater than about 0.5 μM, 1 μM, 2 μM, 5 μM, or 10 μM.

As used herein, the term “specificity for GSK3β” may be used to refer to GSK3 inhibitors that specifically or selectively inhibit GSK3β, relative to other glycogen synthase kinases, such as GSK3a. For example, a GSK3 inhibitor that specifically inhibits GSK3β may have an IC₅₀ (μM) that is lower than an IC₅₀ for another glycogen synthase kinase, such as GSK3a. A GSK3 inhibitor that specifically inhibits GSK3β over another glycogen synthase kinase, such as GSK3a, has an IC₅₀ (μM) for GSK3β that is at least 2-times lower, at least 3-times lower, at least 5-times lower, at least 10-times lower, at least 20-times lower, at least 50-times lower, at least 100-times lower, at least 500-times lower, or at least 1000-times lower, than the IC₅₀ (μM) for the other glycogen synthase kinase, such as GSK3a. In some embodiments, the GSK3 inhibitor has an IC₅₀ for GSK3β of less than about 0.050 μM, 0.040 μM, 0.030 μM, 0.020 μM, or 0.010 μM. In some embodiments, the GSK3 inhibitor has an IC₅₀ for GSK3α of greater than about 0.5 μM, 1 μM, 2 μM, 5 μM, or 10 μM.

As used herein, the term “dual inhibitor” may be used to refer to GSK3 inhibitors that inhibit both of GSK3α and GSK3β. For example, a dual GSK3α/β inhibitor may have an IC₅₀ (μM) for GSK3α which is less than about 0.050 μM, 0.040 μM, 0.030 μM, 0.020 μM, or 0.010 μM and the dual GSK3a/0 inhibitor may have an IC₅₀ (μM) for GSK3β which is less than about 0.050 μM, 0.040 μM, 0.030 μM, 0.020 μM, or 0.010 μM. In some embodiments, the GSK3 inhibitor utilized in the disclosed methods for treating CTNNB1 syndrome is not a dual GSK3a/0 inhibitor (e.g., where the GSK3 inhibitor specifically or selectively inhibits GSK3β or GSK3a).

Pharmaceutically acceptable salts of the disclosed GSK3 inhibitors also are contemplated herein and may be utilized in the disclosed treatment methods. For example, a substituent group of the disclosed GSK3 inhibitors may be protonated or deprotonated and may be present together with an anion or cation, respectively, as a pharmaceutically acceptable salt of the compound. The term “pharmaceutically acceptable salt” as used herein, refers to salts of the GSK3 inhibitors which are substantially non-toxic to living organisms. Typical pharmaceutically acceptable salts include those salts prepared by reaction of the GSK3 inhibitors as disclosed herein with a pharmaceutically acceptable mineral or organic acid or an organic or inorganic base. Such salts are known as acid addition and base addition salts. It will be appreciated by the skilled reader that most or all of the GSK3 inhibitors as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, alpha-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

It should be recognized that the particular counter-ion forming a part of any salt of a GSK3 inhibitor disclosed herein is usually not of a critical nature, so long as the salt as a whole is pharmacologically acceptable and as long as the counterion does not contribute undesired qualities to the salt as a whole. Undesired qualities may include undesirably solubility or toxicity.

It will be further appreciated that the disclosed GSK3 inhibitors can be in equilibrium with various inner salts. For example, inner salts include salts wherein the GSK3 inhibitor includes a deprotonated substituent group and a protonated substituent group.

The disclosed GSK3 inhibitors may be used to prepare and formulate pharmaceutical compositions. As such, also disclosed herein are pharmaceutical compositions comprising an effective amount of any of the GSK3 inhibitors disclosed herein, or pharmaceutically acceptable salts of any of the GSK3 inhibitors disclosed herein, together with a pharmaceutical excipient. In some embodiments, the disclosed GSK3 inhibitors may be used for preparing a medicament for treating a disease or disorder associated with CTNNB1 syndrome, and in particular, a disease or disorder that may be treated with a specific GSK3β inhibitor, GSK3α inhibitor or a dual paralog GSK3α/β inhibitor. As such, the disclosed GSK3 inhibitors may specifically inhibit GSK3β, GSK3a, or selectively inhibit both GSK3β and GSK3a.

The disclosed GSK3 inhibitors may be used to prepare and formulate pharmaceutical compositions for treating diseases that are associated with CTNNB1 syndrome. The disclosed pharmaceutical compositions may be administered to patients in need thereof in methods for treating CTNNB1 syndrome.

The GSK3 inhibitors and pharmaceutical compositions disclosed herein may be administered to a patient in need thereof to treat CTNNB1 syndrome. In some embodiments, the GSK3 inhibitors disclosed herein may be administered at an effective concentration such that the GSK3 inhibitor effectively binds to GSK3β in order to treat CTNNB1 syndrome. In some embodiments, the concentration of the disclosed compounds that is effective for the compound to function as a GSK3β inhibitor is about 0.05-50 μM (or about 0.05-10 μM, or about 0.05-1 μM).

As used herein, a “patient” may be interchangeable with “subject” or “individual” and means an animal, which may be a human or non-human animal, in need of treatment. Suitable patients for the disclosed methods may include, for example mammals, such as humans, monkeys, dogs, cats, horses, rats, and mice. Suitable human patient include, for example, those who have CTNNB1 syndrome or those who have been determined to be at risk for developing CTNNB1 syndrome, or have related disorders with the key molecular cause being reduced beta-catenin levels.

As used herein, a “patient in need of treatment” may include a patient having CTNNB1 syndrome that is responsive to therapy with a GSK3 inhibitor, and specifically a GSK3β inhibitor, GSK3α inhibitor or a dual GSK3a/β inhibitor.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of CTNNB1 syndrome. As such, the methods disclosed herein encompass both therapeutic and prophylactic administration.

As used herein the term “effective amount” refers to the amount or dose of the GSK3 inhibitor, upon single or multiple dose administration to the subject, which provides the desired effect in the subject under diagnosis or treatment. The disclosed methods may include administering an effective amount of the disclosed selective GSK3β, GSK3a, or GSK3a/P dual inhibitors (e.g., as present in a pharmaceutical composition) for treating CTNNB1 syndrome, whereby the effective amount causes inhibition of GSK3α and R together in the patient.

An effective amount can be readily determined by the attending diagnostician, as one skilled in the art, by the use of known techniques and by observing results obtained under analogous circumstances. In determining the effective amount or dose of GSK3 inhibitor administered, a number of factors can be considered by the attending diagnostician, such as: the species of the subject; its size, age, and general health; the degree of involvement or the severity of the disease or disorder involved; the response of the individual patient; the particular compound administered; the mode of administration; the bioavailability characteristics of the preparation administered; the dose regimen selected; the use of concomitant medication; and other relevant circumstances.

In some embodiments, a daily dose of the disclosed GSK3 inhibitors may contain from about 0.01 mg/kg to about 100 mg/kg (such as from about 0.05 mg/kg to about 50 mg/kg and/or from about 0.1 mg/kg to about 25 mg/kg) of the GSK3 inhibitor used in the present method of treatment. The dose may be administered under any suitable regimen (e.g., weekly, daily, twice daily).

The pharmaceutical compositions for use according to the methods as disclosed herein may include a single GSK3 inhibitor (or specifically a GSK3β inhibitor, a GSK3α inhibitor or a GSK3α/β dual paralog inhibitor) as an active ingredient or a combination of GSK3 inhibitors as active ingredients (e.g., a combination of GSK3α and R selective inhibitors). For example, the methods disclosed herein may be practiced using a composition containing a single compound that is a GSK3α/β dual paralog inhibitor. Alternatively, the disclosed methods may be practiced using a composition containing two or more compounds that are GSK3β inhibitors, or combinations of selective GSK3α inhibitors and GSK3β inhibitors.

Instead of administering a pharmaceutical composition comprising two or more compounds that are GSK3 inhibitors, the disclosed methods may be practiced by administering a first pharmaceutical composition (e.g., a pharmaceutical composition comprising a GSK3β inhibitor) and administering a second pharmaceutical composition (e.g., a pharmaceutical composition comprising a different GSK3β and/or GSK3α inhibitor), where the first composition may be administered before, concurrently with, or after the second composition. As such, the first pharmaceutical composition and the second pharmaceutical composition may be administered concurrently or in any order, irrespective of their names.

As one skilled in the art will also appreciate, the disclosed pharmaceutical compositions can be prepared with materials (e.g., actives excipients, carriers, and diluents etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The GSK3 inhibitors utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. Alternatively, the GSK3 inhibitors utilized in the methods disclosed herein may be formulated as a pharmaceutical composition in liquid form (e.g., an injectable liquid or gel)

The GSK3 inhibitors utilized in the methods disclosed herein may be formulated as a pharmaceutical composition that includes an excipient, carrier, or diluent. For example, the excipient, carrier, or diluent may be selected from the group consisting of proteins, carbohydrates, sugar, talc, magnesium stearate, cellulose, calcium carbonate, and starch-gelatin paste.

The GSK3 inhibitors utilized in the methods disclosed herein also may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents for the pharmaceutical compositions may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

The disclosed pharmaceutical compositions also may include disintegrants. Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

The disclosed pharmaceutical compositions also may include effervescent agents. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the GSK3β, GSK3α or dual GSK3α/β inhibitor may be employed with either a paraffinic or a water-miscible ointment base. Alternatively, the GSK3β, GSK3α or dual GSK3α/β inhibitor may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

### Examples

The following examples are illustrative and should not be interpreted to limit the claimed subject matter.

Example 1. Identifying molecular and functional changes and treatment strategies for CTNNB1 syndrome—small molecule GSK3α and GSK3β inhibitors being tested as drug treatments for CTNNB1 syndrome.

The small molecule GSK3α and GSK3β inhibitors discussed herein are disclosed in Wagner et al. 2018, Sci Transl Med. 10(431).

CTNNB1 syndrome is a human developmental disorder characterized by intellectual disabilities, microcephaly, motor and speech delays, truncal hypotonia, peripheral hypertonia, spasticity, mild visual defects. It is caused by CTNNB1 (beta-catenin) haploinsufficiency due to partial or complete deletion mutations. CTNNB1 is a high-confidence risk gene for intellectual disabilities. Treatments for CTNNB1 Syndrome are lacking due to limited knowledge of the underlying pathophysiological changes and limited studies of in vivo mouse and in vitro human cell models of CTNNB1 haploinsufficiency.

As such, research studies were performed to address these limitations. Two different preclinical in vivo mouse and in vitro human cell models were generated and characterized. Small molecule GSK3α and GSK3β inhibitors were tested for drug treatments for amelioration.

To investigate CTNNB1 syndrome, a new mouse model with full body deletion of one CTNNB1 allele has been generated. The CTNNB1/β-catenin heterozygous mouse line, or βcatHet mice, have been used to (1) assess for recapitulation of key features of CTNNB1 syndrome (e.g. learning and motor skills), (2) define the molecular and functional underpinnings, and (3) test drug treatments, in vivo, for safe and effective amelioration of the phenotypes.

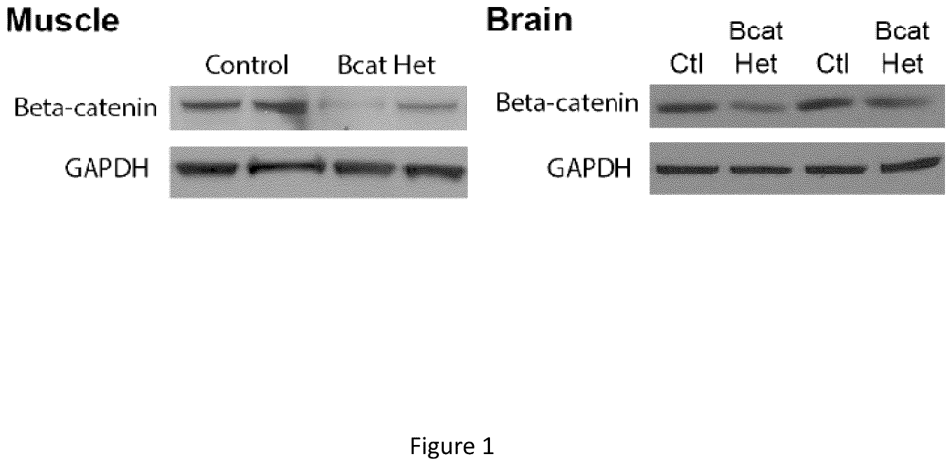

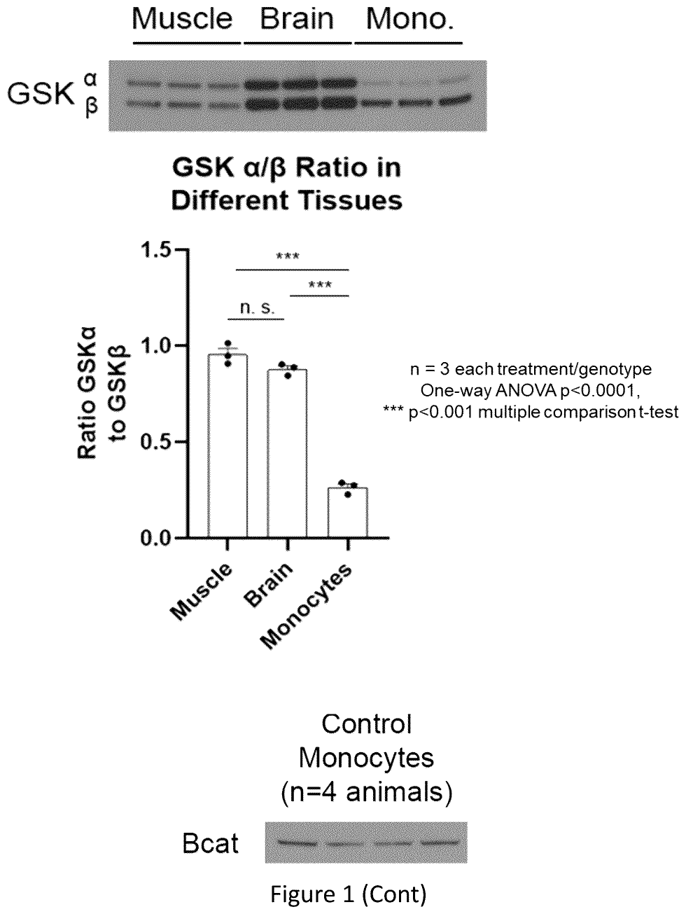

In the new βcatHet mice, β-catenin protein and mRNA levels decrease by 50% in β-catenin heterozygous mice, relative to littermate controls as evidenced by quantitative experiments. To determine which tissues to test for GSK3, muscle, brain, and monocytes were examined in the same way. While present in all three samples, the GSKα: GSKβratio in monocytes is different from the ratio in muscle and brain; therefore muscle and brain were used for the inhibitor studies. Additionally, muscle and brain are disease relevant tissues in CTNNB1 syndrome See FIG. 1.

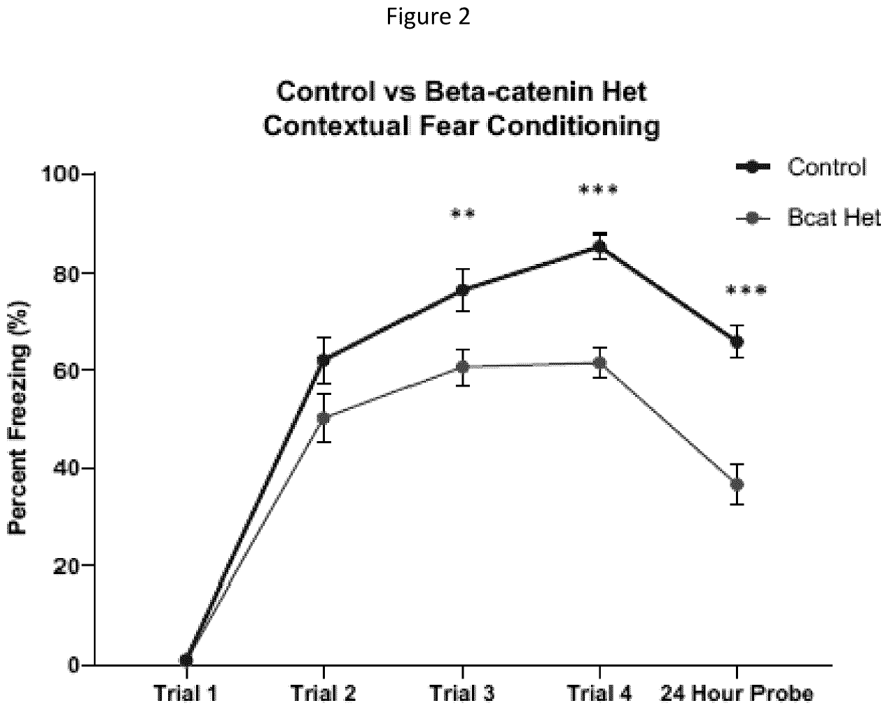

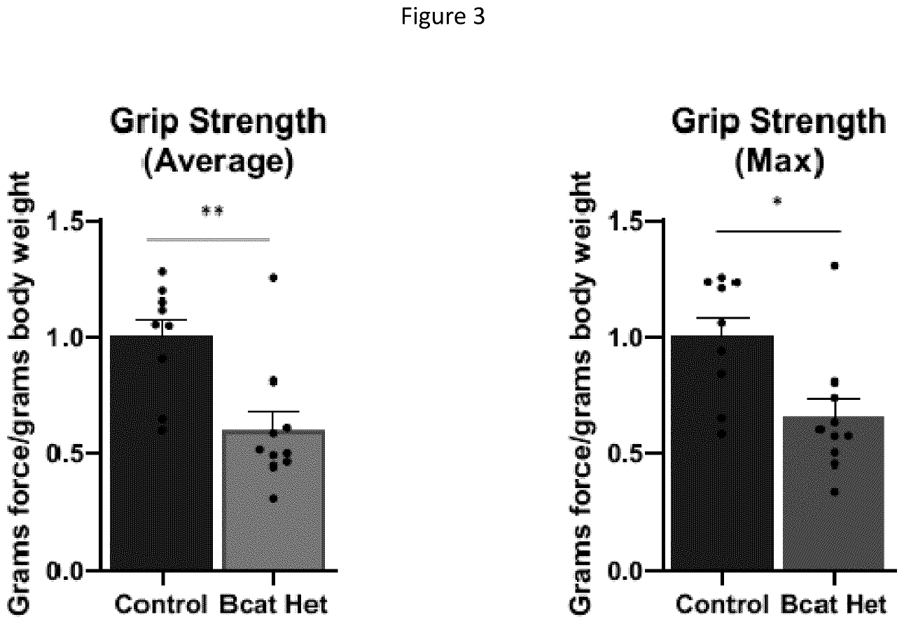

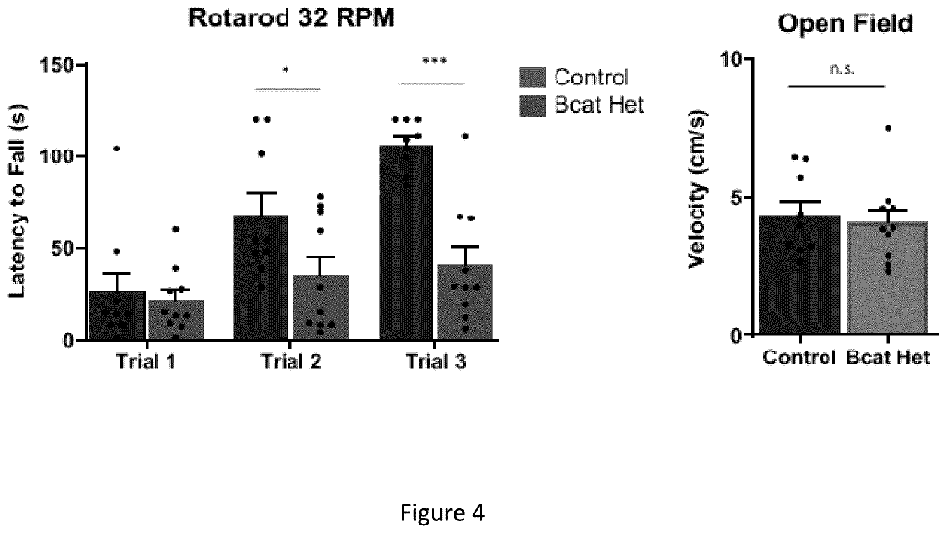

The β-catenin heterozygous mouse line displays functional changes that resemble CTNNB1 syndrome, establishing it as a powerful preclinical in vivo model for testing drug treatments to correct the disabilities. The β-catenin heterozygous mice exhibit reduced associative learning (for example, contextual fear conditioning), relative to littermate controls. See FIG. 2. Moreover, the β-catenin heterozygous mice exhibit reduced muscle grip strength, both average and maximum, relative to littermate controls. See FIG. 3. Additionally, the β-catenin heterozygous mice exhibit reduced motor learning, but no differences in velocity of movement (or distance travelled). See FIG. 4.

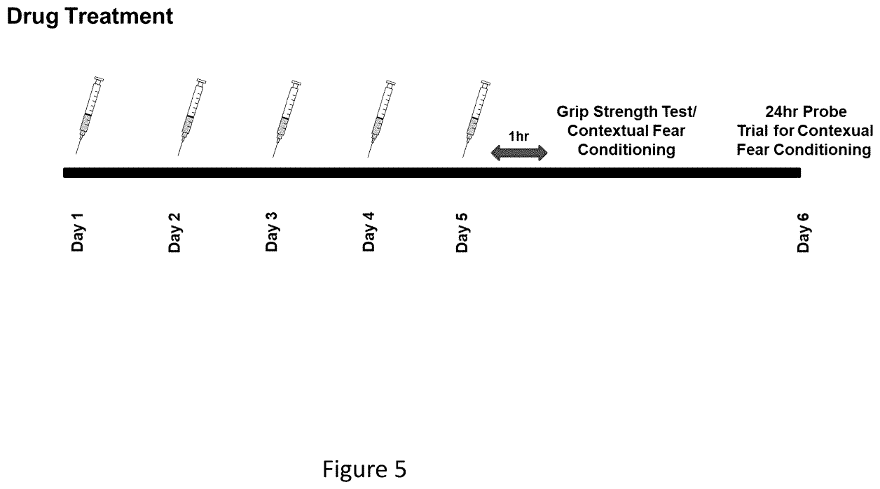

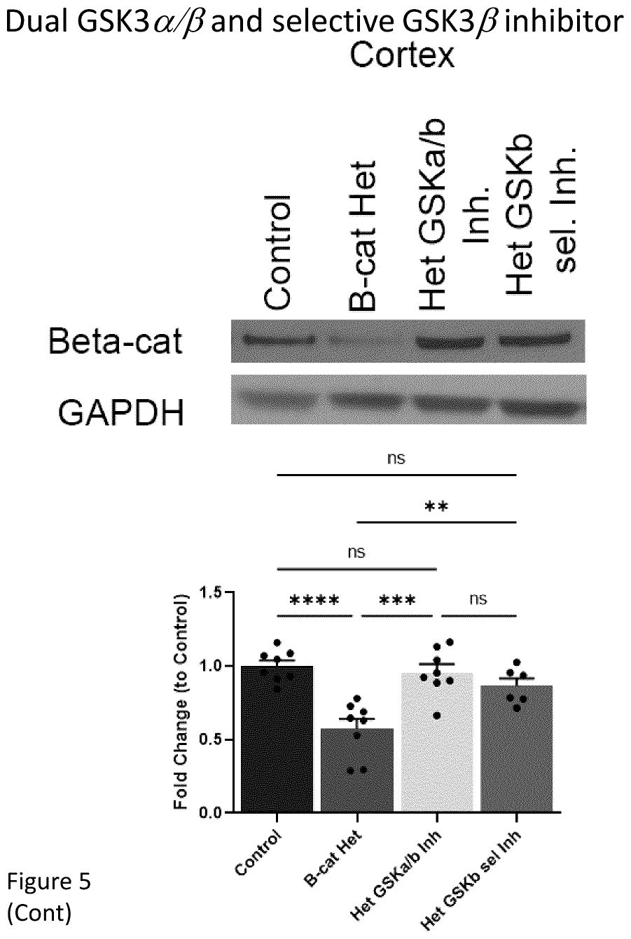

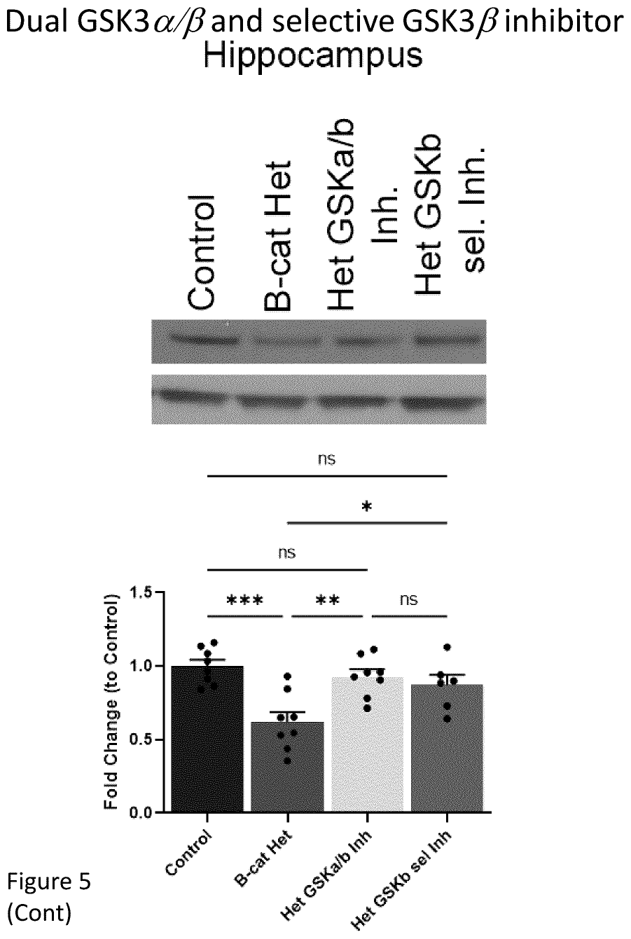

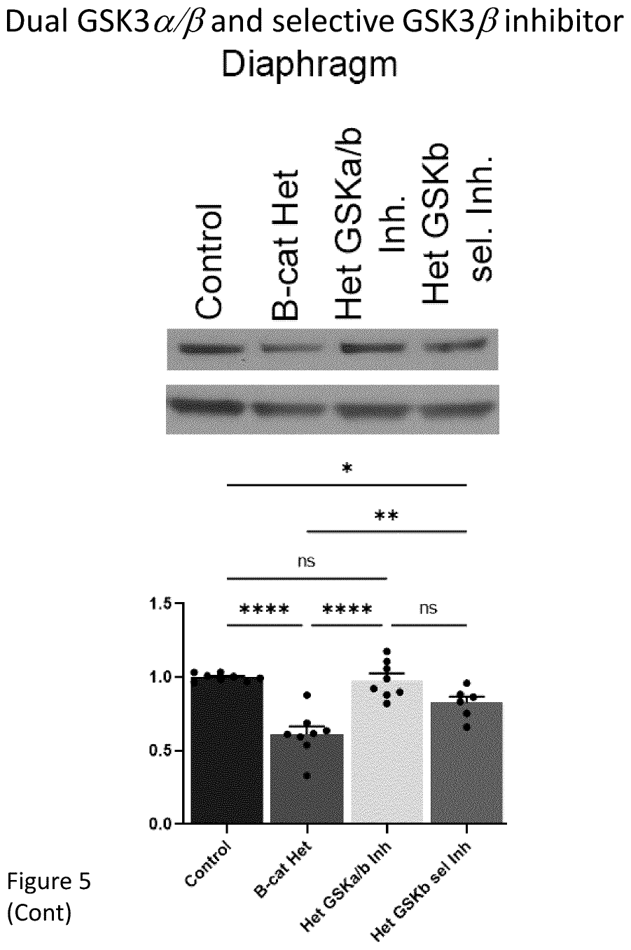

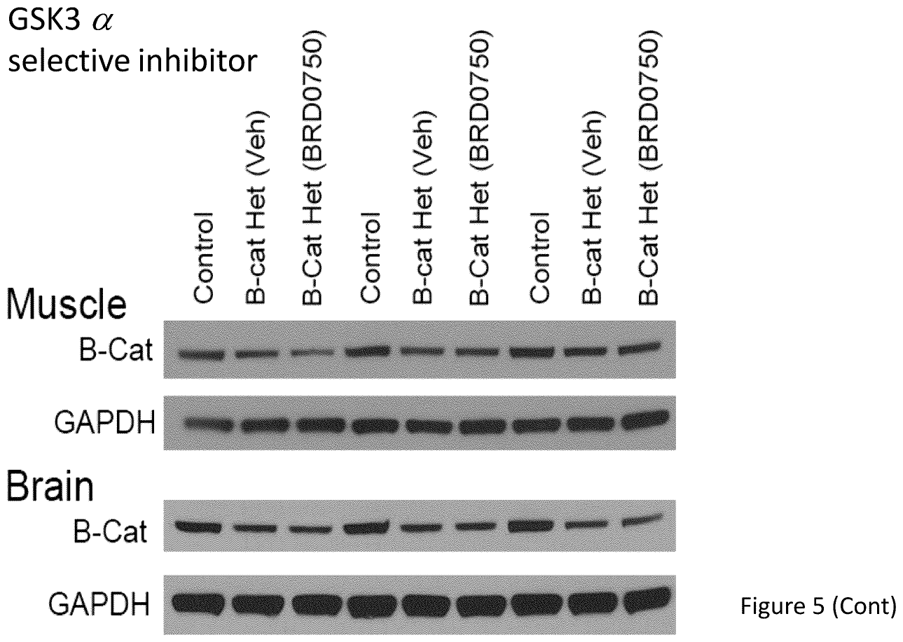

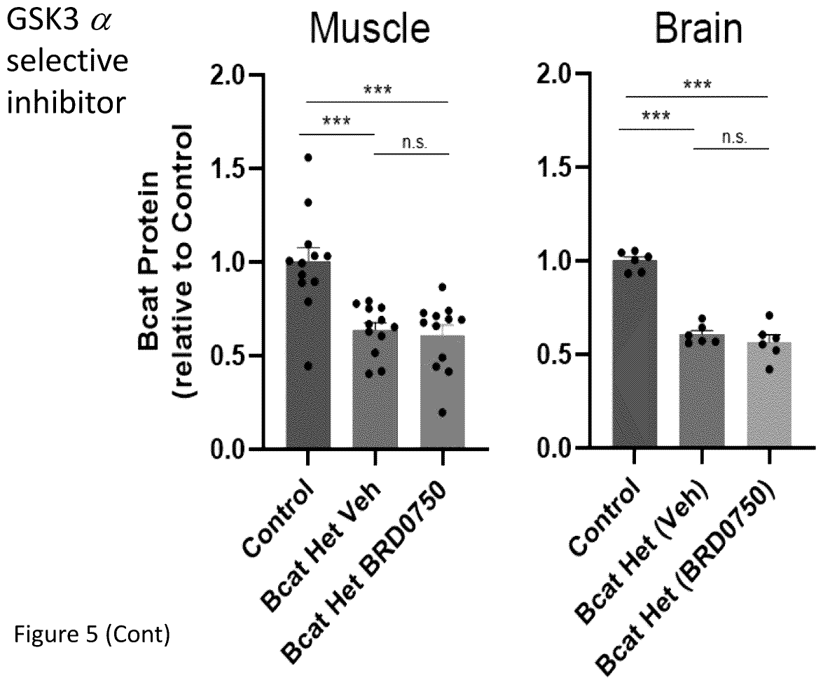

Drug treatments using small molecule GSK3α and GSK3β inhibitors were tested in vivo, for safe and effective amelioration of the phenotypes by assaying β-catenin protein levels, learning and motor skills effects of drug vs. vehicle on CTNNB1 heterozygote mice and control littermates. In an example, a study includes administering the small molecule GSK3 inhibitor in sequential daily doses to the βcatHet mice. The daily doses can be administered, for example, for five days, or four days, or any other number of days. At some time after the last dose, such as 1 hour, the grip strength test can be administered. Additionally, the contextual fear conditioning can be performed. Sometime later, such as 1 day later, the contextual fear conditioning tests can be performed. See FIGS. 5-11.

GSK3 isoform topological differences are driven via the Asp→Glu “switch” within the hinge domain. The pyrazolo-tetrahydroquinolinone-based hinge binders formed a direct H-bond to the analogous hinge position (Asp133 or Glu196 residue of GSK3β or GSK3α respectively). This difference was utilized to generate paralog selective inhibitors, for example GSK3α/β dual paralog inhibitor BRD0320, GSK3α selective inhibitor BRD0705 and GSK3β selective inhibitor BRD3731. The tridentate binding mode of the core scaffold provides a rigid molecular platform within the ATP binding domain well suited to explore apparent differences within the hydrophobic selectivity pockets. These differences were systematically probed by designing inhibitors predicted to be preferential binders for either GSK3α or GSK3β. See Wagner et al. 2018, Sci Transl Med. 10(431).

The small molecule inhibitors BRD0320 (Drug 1), BRD0705 (Drug 2) and BRD3731 were tested in the in vivo mouse model of CTNNB1 syndrome disclosed here. Mice were administered 30 mg/kg doses of the respective compounds in the present Example. Samples of the brain and muscle from control mice, βcatHet mice (untreated), and βcatHet mice (treated) were tested for levels of β-catenin. In samples from βcatHet mice treated with GSK3α/β dual paralog inhibitor BRD0320, significantly corrected β-catenin protein levels are observed as evidenced by the gel data shown in FIG. 5.

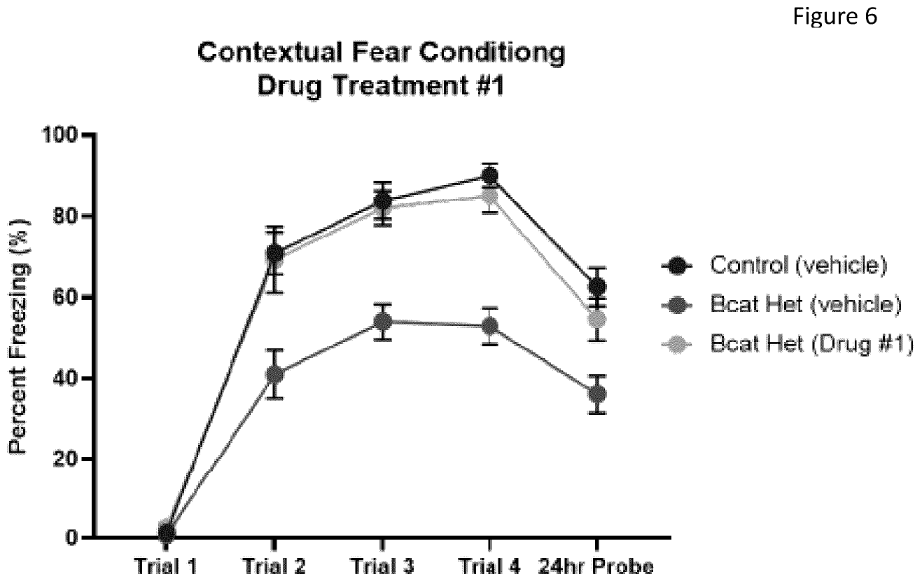

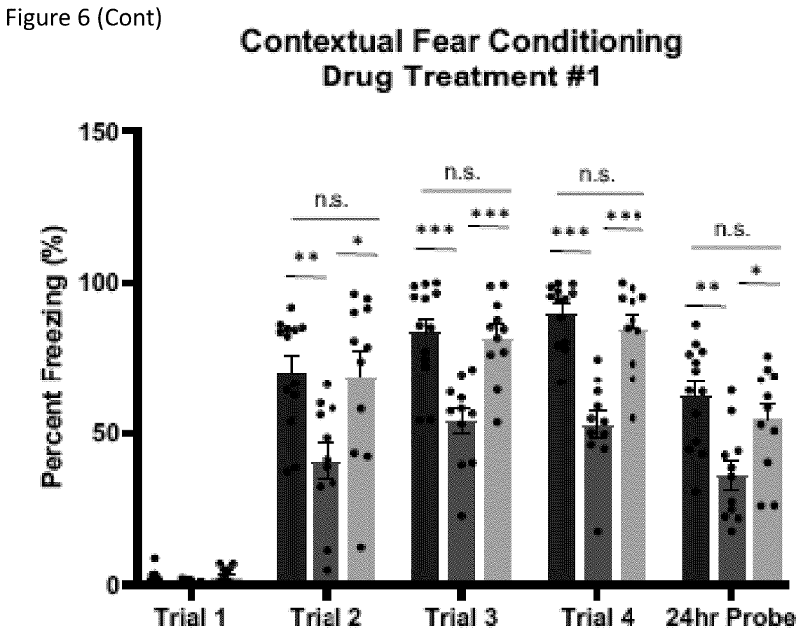

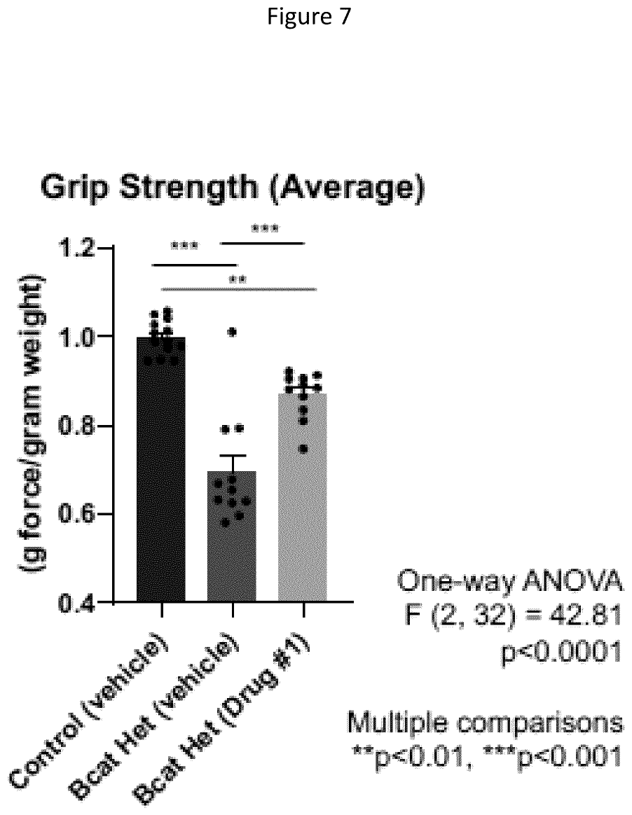

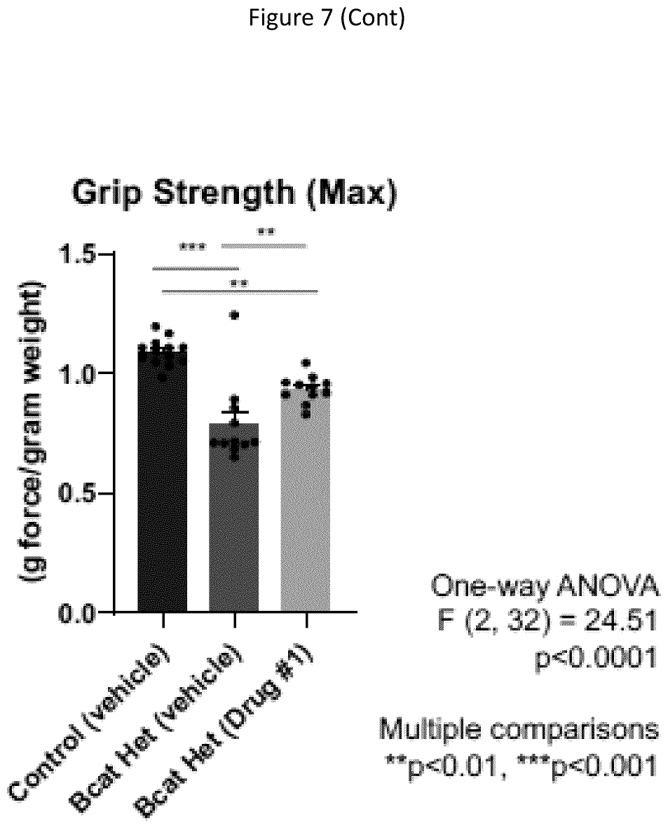

BRD0320 treatment significantly corrected cognitive disabilities in βcatHet mice as shown by contextual fear conditioning test results (FIG. 6) as well as significantly improving motor disabilities, for example, grip strength, as shown in FIG. 7.

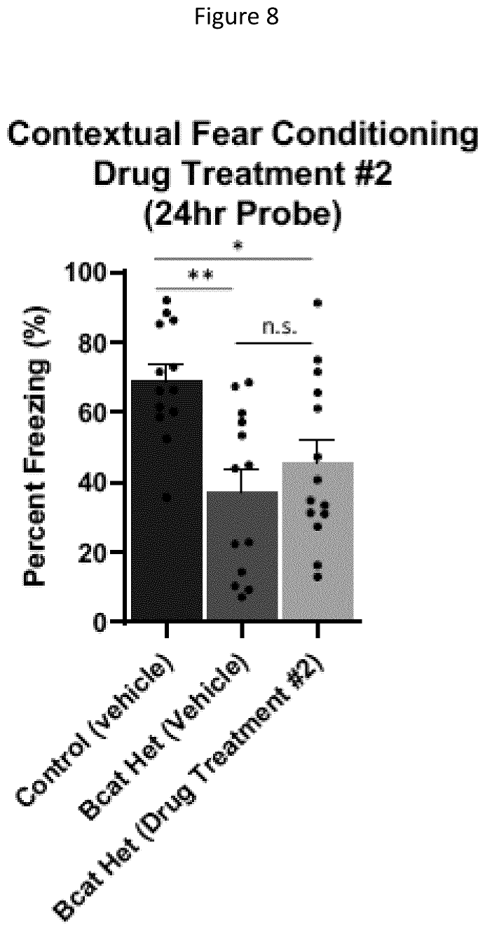

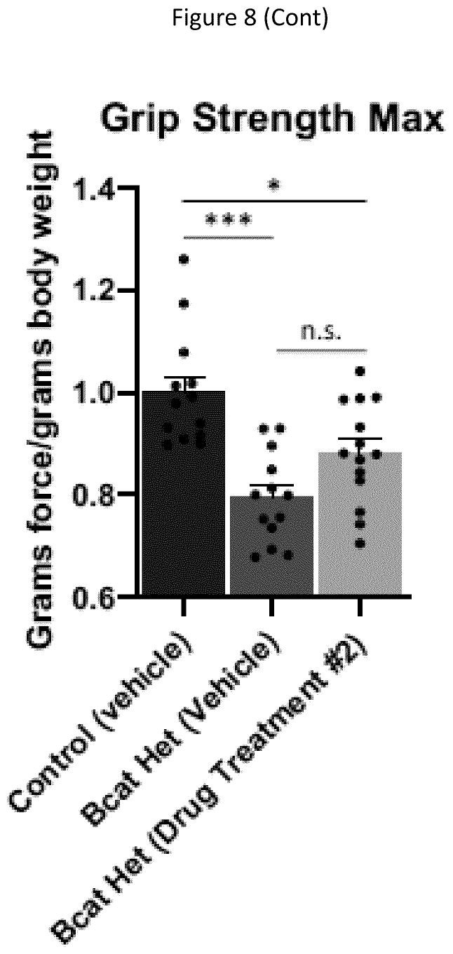

The GSK3α selective inhibitor BRD0705 (Drug 2) did not display a significant trend to improvements in learning and grip strength. Levels of β-catenin were not significantly different from those found in untreated βcatHet mice. See FIGS. 5, 8.

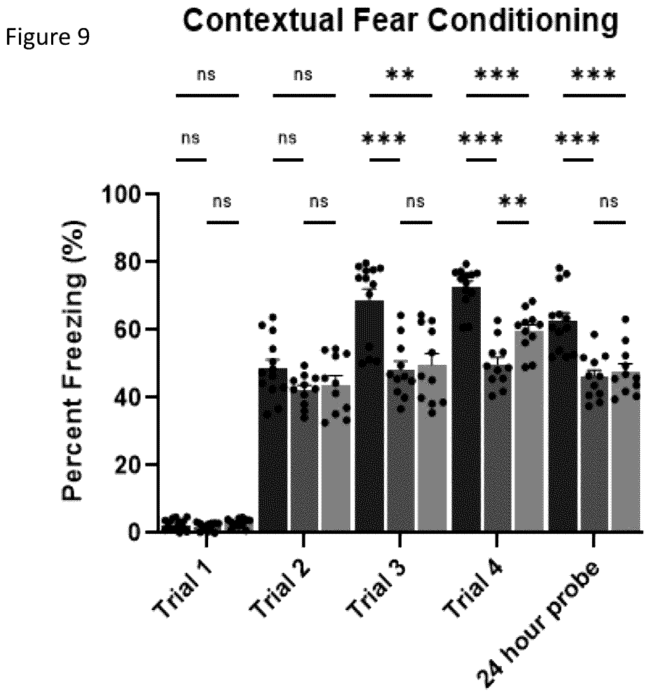

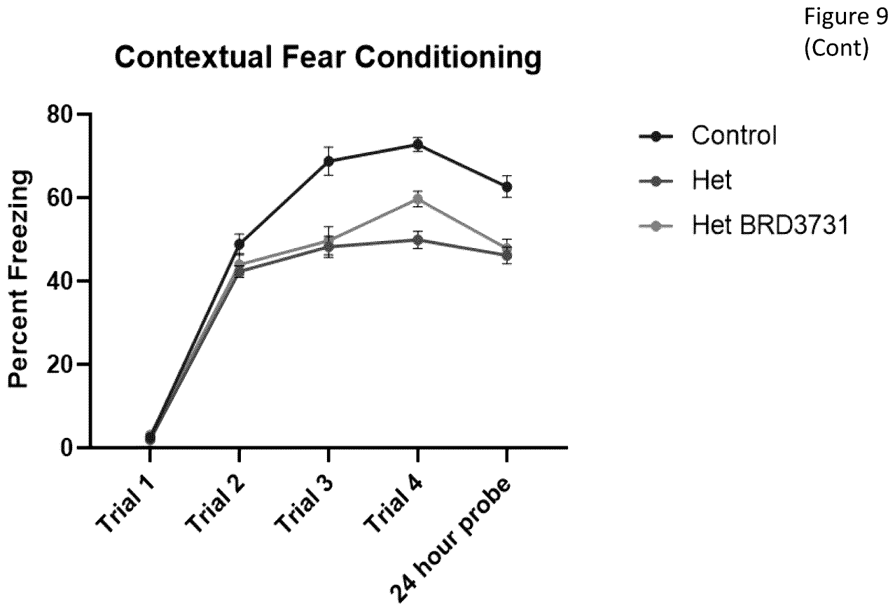

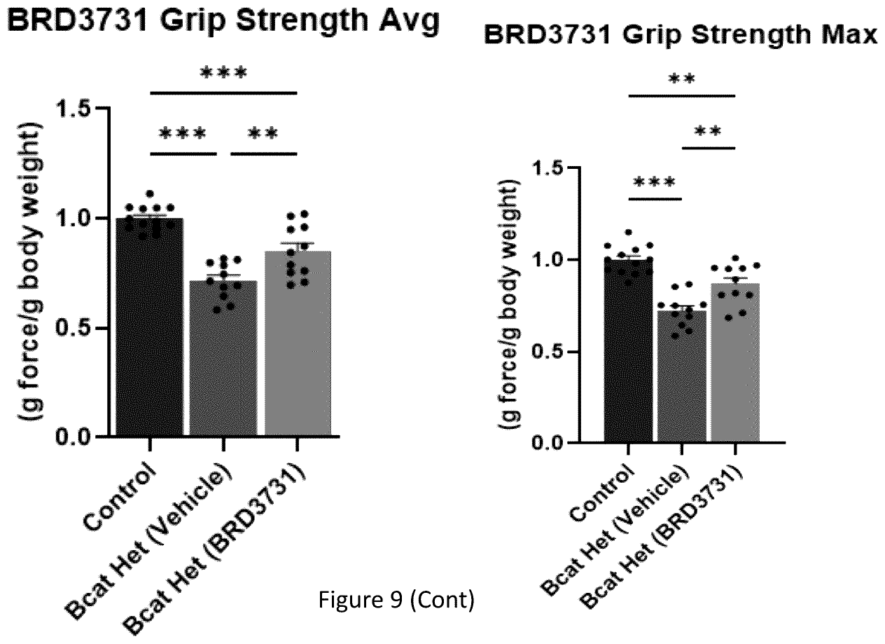

The GSK3β selective inhibitor BRD3731 significantly increased β-catenin protein levels as shown by quantitative immunoblotting. See FIG. 5. BRD3731 provided slight significant improvement in learning as well as a significant increase in muscle grip strength. See FIG. 9.

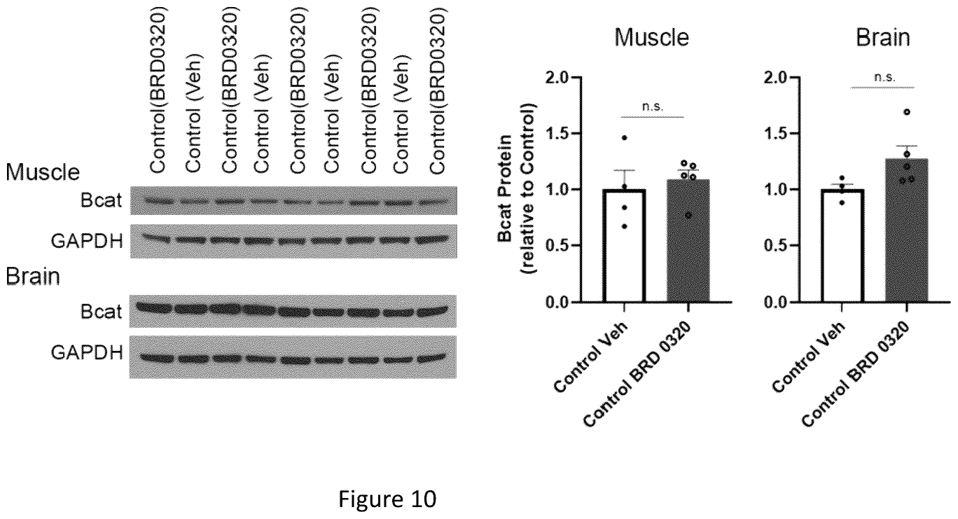

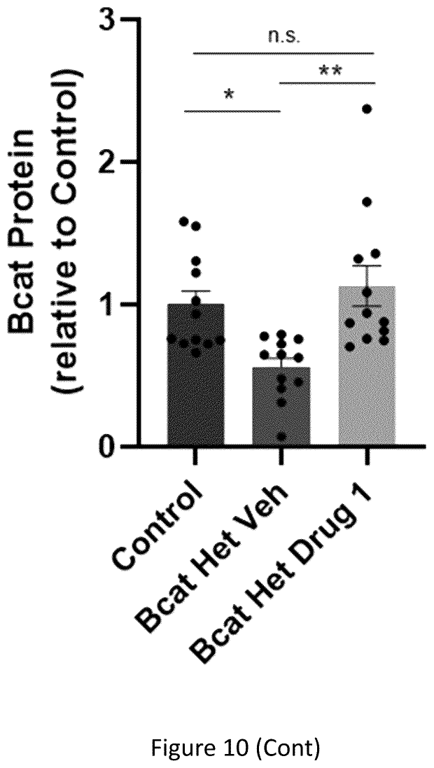

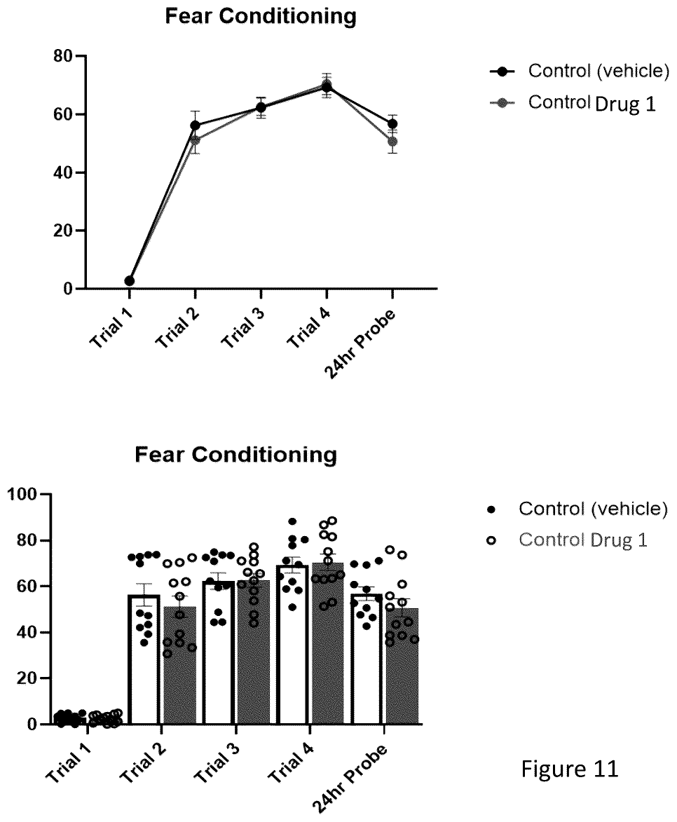

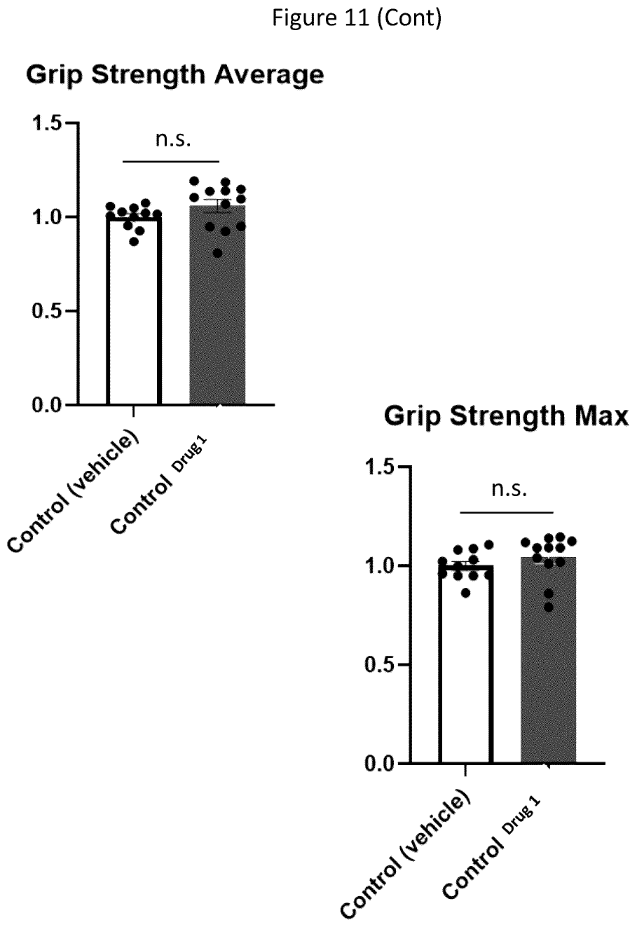

The GSK3α/β dual paralog inhibitor BRD0320 (Drug 1) does not cause adverse changes in wildtype littermate mice treated with the same paradigm as the βcatHet mice, based on no significant changes in β-catenin protein levels (FIG. 10). Additionally, administration of the GSK3α/β inhibitor does not increase 3-cat levels in βcatHet mice above baseline wildtype levels. See FIG. 10. No significant changes are observed for learning or muscle grip strength in drug-treated control wildtype mice relative to vehicle-treated control wildtype mice (FIG. 11). See FIGS. 10,11. The treatment of βcatHet mice with GSK3α/β dual inhibitor BRD0320 has provided very promising results showing improved learning and motor capabilities with in vivo drug treatment at young adult age. Neither the GSK3α selective inhibitor BRD0705 nor the GSK3β selective inhibitor BRD3731 are as effective at correcting the cognitive and motor disabilities as the GSK3α/β dual inhibitor BRD0320, suggesting that the combined inhibition of both GSK3 isoforms is required.

Example 2. Identifying structures for small molecule GSK3α and GSK3β inhibitors for testing as drug treatments for CTNNB1 syndrome.

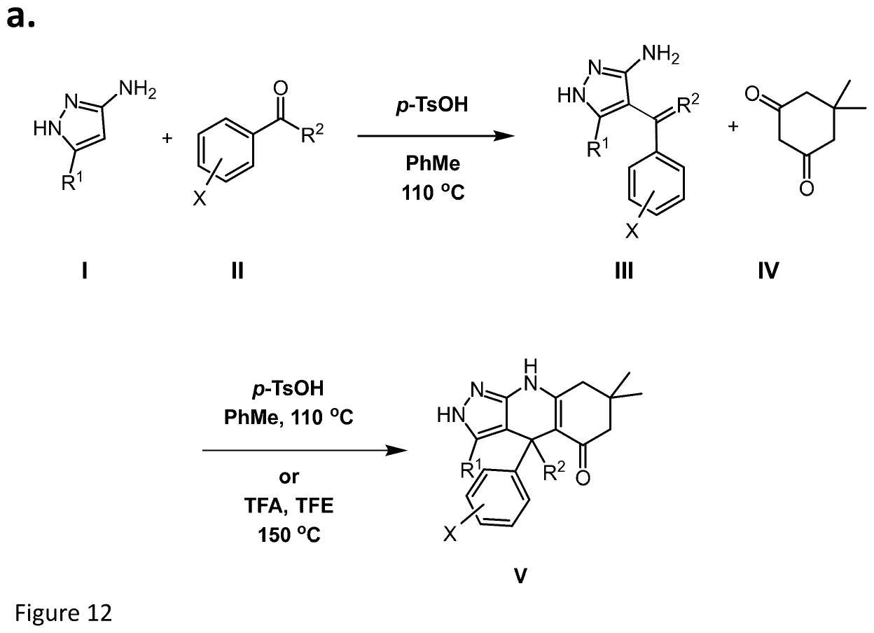

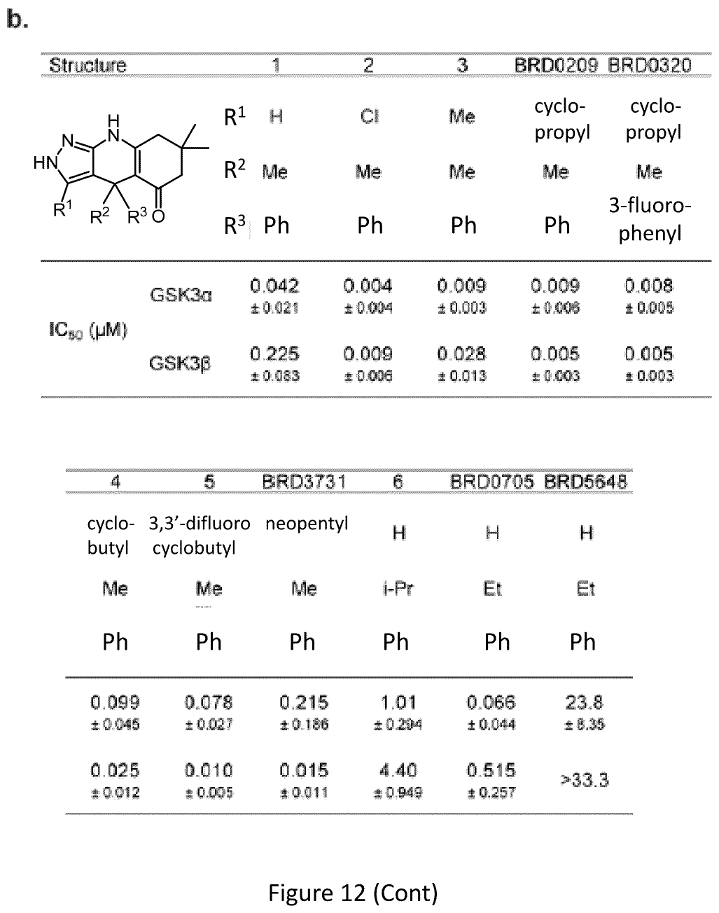

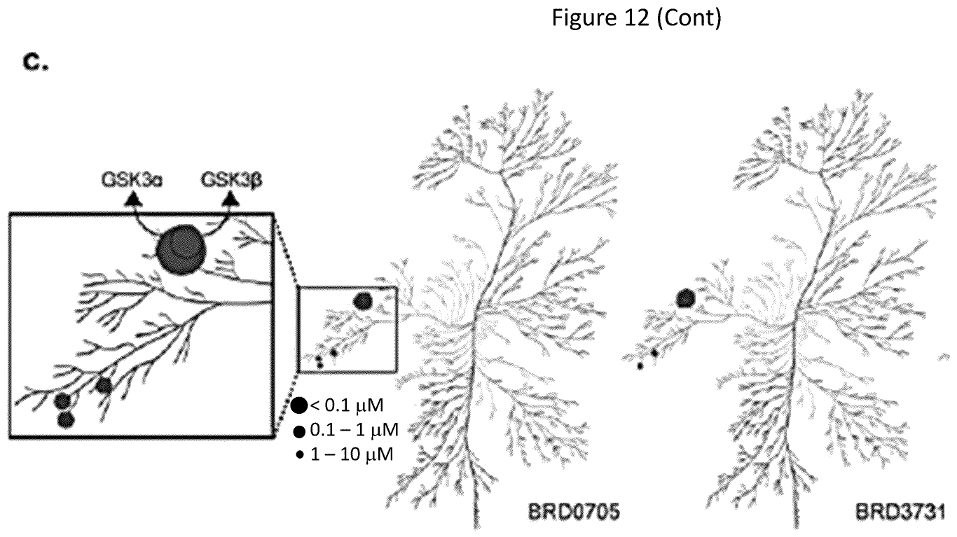

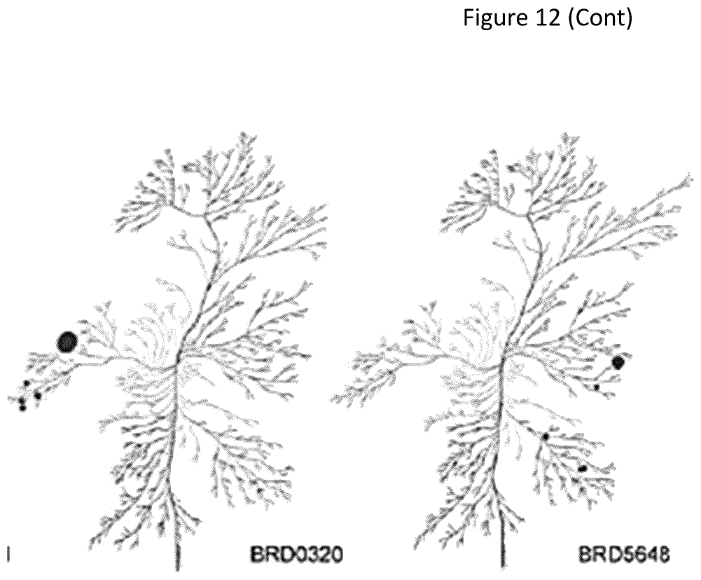

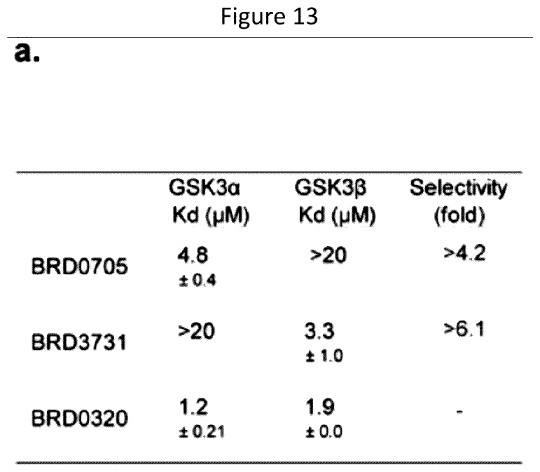

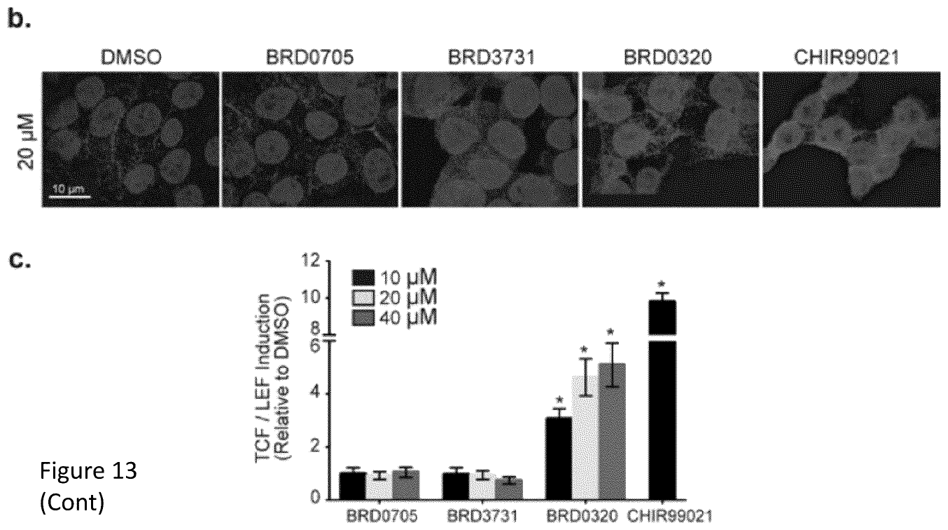

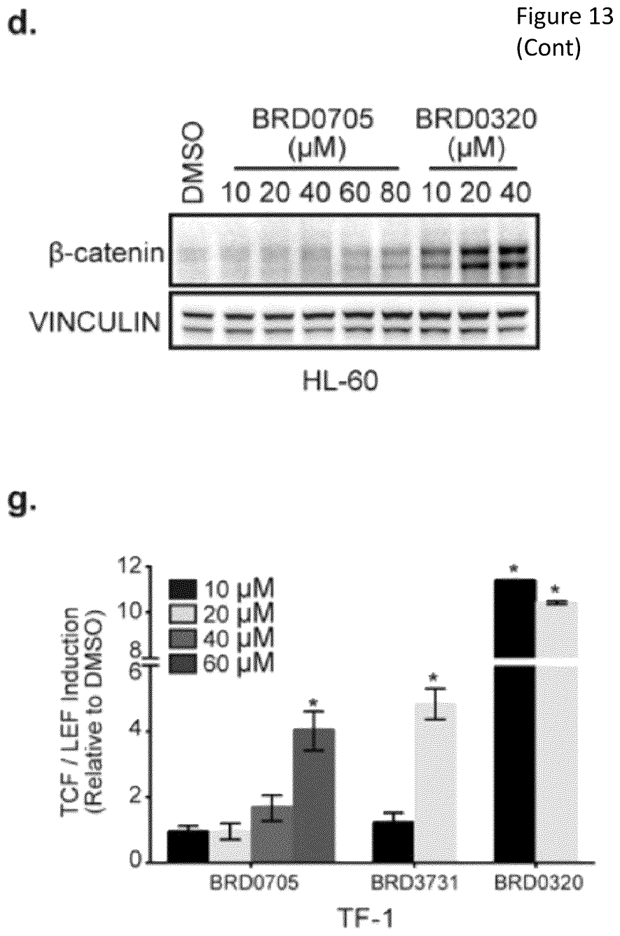

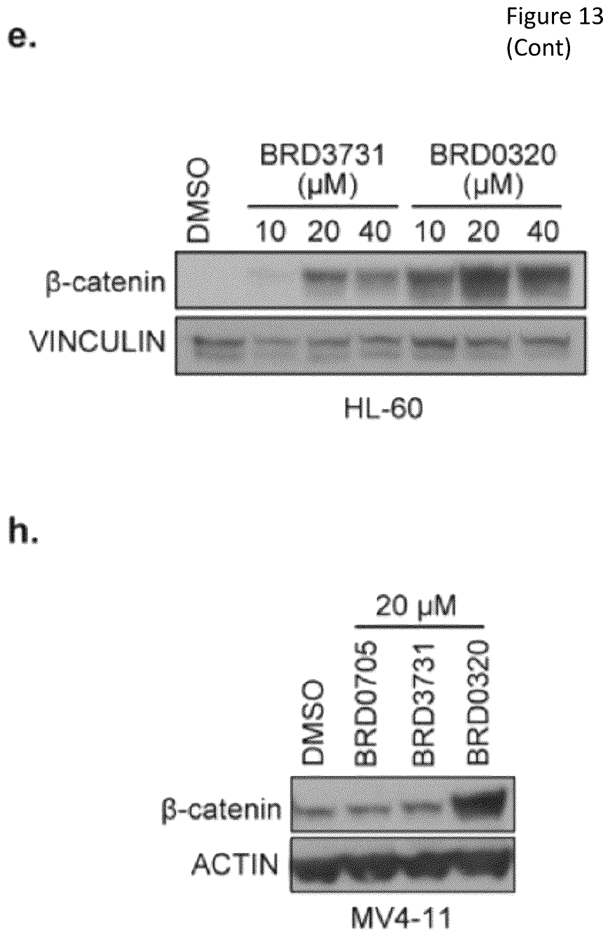

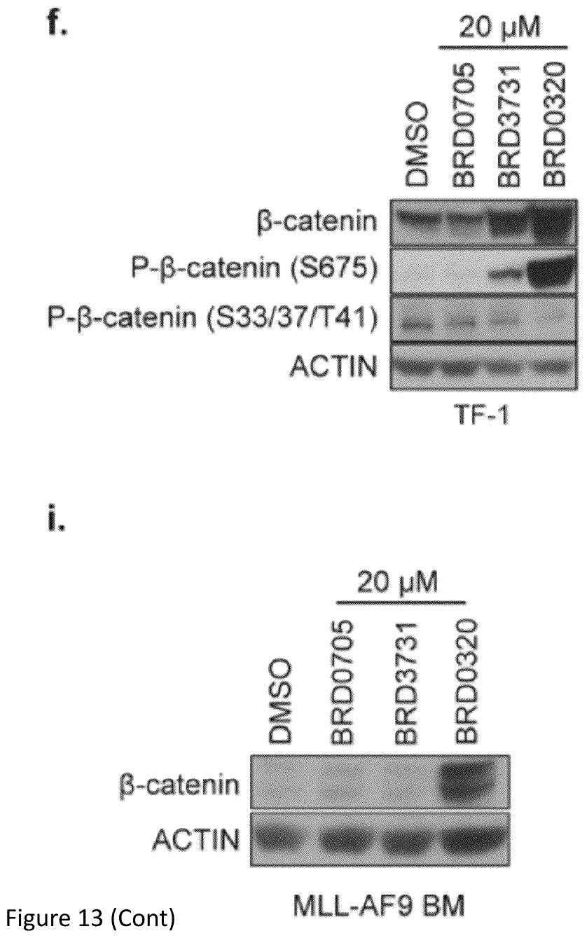

Structure-based design has been aimed towards maximizing steric requirement at the R position and the corresponding differences within the selectivity pockets of GSK3α and GSK3β. See FIG. 12. An ethyl substitution (BRD0705) maintained potency and displayed increased selectivity for GSK3α (8-fold) versus GSK3β (GSK3α IC₅₀=66 nM; GSK3 IC₅₀=515 nM). Neopentyl substituted compound BRD3731, displayed 14-fold selectivity for GSK3β (GSK3β IC₅₀=15 nM; GSK3α IC₅₀=215 nM). Equipotency for the two paralogs was observed with electronically neutral, but increasingly bulkier substituents: a cyclopropyl in BRD0209, implicating hydrophobic rather than electronic effects. These modifications and increased binding affinities were tolerant of simple substitutions on the phenyl ring (cf. BRD0320). See FIG. 13.

The studies provide critical proof-of-concept for pharmacological rescue, in vivo, in a relevant genetic model of CTNNB1 haploinsufficiency—the CTNNB1 heterozygous mouse.

Example 3. Effects of GSK3 inhibitors on beta-catenin levels in CTNNB1 heterozygous neurons

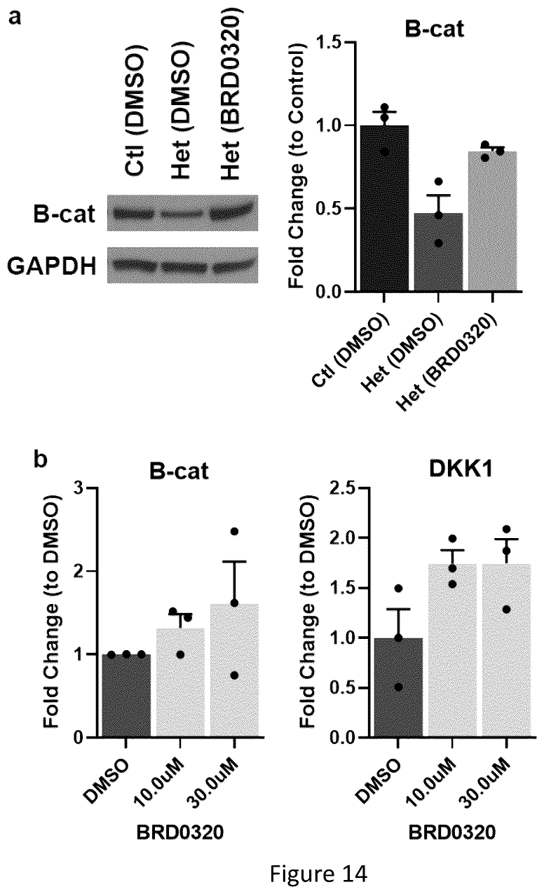

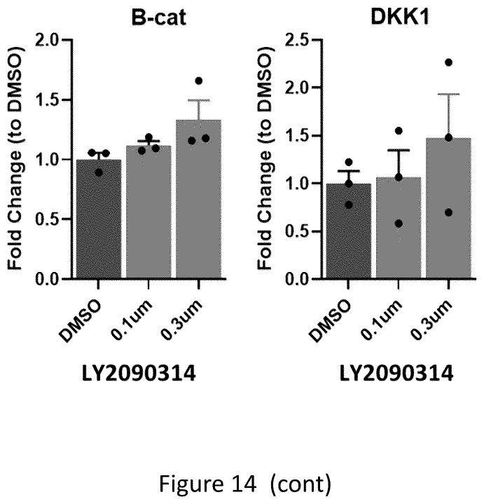

A human pluripotent stem cell line (iPSC) that lacks any pathogenic variants was made heterozygous for CTNNB1 via CRISPR-Cas9 gene editing of one allele. A premature termination (PTC+1) frameshift was introduced based on a CTNNB1 syndrome patient mutation. Haploinsufficiency was confirmed. The iPSCs were differentiated to cortical glutamatergic neurons by transfection with the transcription factor human neurogenin 2. The glutamatergic neurons at 21 days in vitro were treated with the GSK3 inhibitor BRD0320 at 30 μM. As shown in FIG. 14 (a), beta-catenin protein levels were measured 24 hrs later by quantitative immunoblotting in the GSK3 inhibitor treated CTNNB1 heterozygous neurons, relative to vehicle-treated heterozygous neurons and wildtype neurons.

FIG. 14 (b) shows pilot dose-response studies of CTNNB1 heterozygous iPSCs treated with the different GSK3 inhibitors at the indicated concentrations (BRD0320, 10 μM, 30 μM; LY2090314, 0.1 μM, 0.3 μM). Beta-catenin protein levels were measured 24 hrs later by quantitative immunoblotting in the GSK3 inhibitor treated CTNNB1 heterozygous iPSCs, relative to vehicle-treated heterozygous iPSCs.

We claim:

1. A method for treating CTNNB1 syndrome in a subject in need thereof, the method comprising administering to the subject an effective amount of one or more GSK3 inhibitors, or a pharmaceutically acceptable salt thereof, or a pharmaceutical composition comprising an effective amount of the one or more GSK3 inhibitors, or a pharmaceutically acceptable salt thereof, and a pharmaceutical excipient, carrier, or diluent.

2. The method of claim 1, wherein the one or more GSK3 inhibitors comprise a compound having an IC₅₀ for GSK3α of less than about 0.050 μM.

3. The method of claim 1, wherein the one or more GSK3 inhibitors comprise a compound having an IC₅₀ for GSK3β of less than about 0.050 μM.

4. The method of claim 1, wherein the one or more GSK3 inhibitors comprise a compound having an IC₅₀ for GSK3α of less than about 0.050 μM and an IC₅₀ for GSK3β of less than about 0.050 μM.

5. The method of claim 1, wherein the one or more GSK3 inhibitors comprise a first compound having an IC₅₀ for GSK3α of less than about 0.050 μM and a second compound having an IC₅₀ for GSK3β of less than about 0.050 μM that is different than the first compound.

6. The method of claim 1, wherein the one or more GSK3 inhibitors comprise a compound of Formula I: 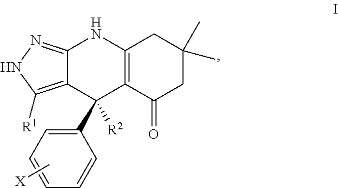
wherein X is hydrogen or halogen;
R¹ is alkyl, unsubstituted or substituted cycloalkyl optionally substituted at one or more positions with halogen, or halogen; and
R² is alkyl.

7. The method of claim 6, wherein X is hydrogen.

8. The method of claim 6, wherein R¹ is the unsubstituted or substituted cycloalkyl optionally substituted at one or more positions with halogen.

9. The method of claim 8, wherein R¹ is cyclopropyl.

10. The method of claim 9, wherein R¹ is the alkyl.

11. The method of claim 10, wherein R¹ is methyl.

12. The method of claim 6, wherein R¹ is halogen.

13. The method of claim 12, wherein R¹ is chloro.

14. The method of claim 6, wherein R² is methyl.

15. The method of claim 6, wherein X is hydrogen, R¹ is cyclopropyl, and R² is methyl.

16. The method of claim 6, wherein X is fluoro, R¹ is cyclopropyl, and R² is methyl.

17. The method of claim 6, wherein X is hydrogen, R¹ is chloro, and R² is methyl.

18. The method of claim 6, wherein X is hydrogen, R¹ is methyl, and R² is methyl.

19. The method of claim 1, wherein the one or more GSK3 inhibitors comprise a compound selected from the group consisting of N-(3-(1H-1,2,4-triazol-1-yl)propyl)-5-(3-chloro-4-methoxyphenyl)oxazole-4-carboxamide, 3-(5-fluorobenzofuran-3-yl)-4-(5-methyl-5H-[1,3]dioxolo[4,5-f]indol-7-yl)-1H-pyrrole-2,5-dione, 2-hydroxy-3-(5-(morpholinomethyl)pyridin-2-yl)-1H-indole-5-carbonitrile, 3-amino-6-(4-(3-((3-methoxypropyl)amino)propyl)phenyl)-N-(pyridin-3-yl)pyrazine-2-carboxamide, 3-amino-6-(4-((4-methylpiperazin-1-yl)sulfonyl)phenyl)-N-(pyridin-3-yl)pyrazine-2-carboxamide, 5-fluoro-N-(3-fluoro-4-(methylsulfonyl)phenyl)-4-(1-(tetrahydro-2H-pyran-4-yl)-1H-imidazol-5-yl)pyrimidin-2-amine, N-(4-(4-chlorophenyl)pyridin-3-yl)-2-(cyclopropanecarboxamido)isonicotinamide, 2-((4-cyanophenyl)amino)-N-(4-(4-fluorophenyl)pyridin-3-yl)pyrimidine-4-carboxamide, 2-((4-fluorophenyl)amino)-N-(4-(4-fluorophenyl)pyridin-3-yl)pyrimidine-4-carboxamide, 2-((4-(difluoromethoxy)phenyl)amino)-N-(4-(4-fluorophenyl)pyridin-3-yl)pyrimidine-4-carboxamide, (S)-2-(2-(4-fluorophenyl)morpholino)-1-methyl-[4,4′-bipyrimidin]-6(1H)-one, 4-benzyl-2-(naphthalen-1-yl)-1,2,4-thiadiazolidine-3,5-dione, 6-((2-((4-(2,4-dichlorophenyl)-5-(5-methyl-1H-imidazol-2-yl)pyrimidin-2-yl)amino)ethyl)amino)nicotinonitrile, N2-(2-((4-(2,4-dichlorophenyl)-5-(1H-imidazol-1-yl)pyrimidin-2-yl)amino)ethyl)-5-nitropyridine-2,6-diamine, 4-benzyl-2-methyl-1,2,4-thiadiazolidine-3,5-dione, 1-(4-methoxybenzyl)-3-(5-nitrothiazol-2-yl)urea, 3-((3-chloro-4-hydroxyphenyl)amino)-4-(2-nitrophenyl)-1H-pyrrole-2,5-dione, 3-(2,4-dichlorophenyl)-4-(1-methyl-1H-indol-3-yl)-1H-pyrrole-2,5-dione, 3-((6-(3-aminophenyl)-7H-pyrrolo[2,3-d]pyrimidin-4-yl)oxy)phenol, 9-bromo-7,12-dihydropyrido[3′,2′:2,3]azepino[4,5-b]indol-6(5H)-one, 9-bromo-7,12-dihydrobenzo[2,3]azepino[4,5-b]indol-6(5H)-one, 2-methyl-5-(3-(4-(methylsulfinyl)phenyl)benzofuran-5-yl)-1,3,4-oxadiazole, and 3-amino-6-(4-((4-(4-(1-(17-((2-(2,6-dioxopiperidin-3-yl)-1,3-dioxoisoindolin-4-yl)amino)-3,6,9,12,15-pentaoxaheptadecyl)-1H-1,2,3-triazol-4-yl)butyl)piperazin-1-yl)sulfonyl)phenyl)-N-(pyridin-3-yl)pyrazine-2-carboxamide.

20. A method for increasing β-catenin protein levels in a subject in need thereof, the method comprising administering to the subject an effective amount of one or more GSK3 inhibitors, or a pharmaceutically acceptable salt thereof, or a pharmaceutical composition comprising an effective amount of the one or more GSK3 inhibitors, or a pharmaceutically acceptable salt thereof, and a pharmaceutical excipient, carrier, or diluent.

21. The method of claim 20, wherein the subject has a complete deletion, partial deletion or mutation of the CTNNB1 gene.

22. The method of claim 20, wherein the one or more GSK3 inhibitors comprise
(a) a compound having an IC₅₀ for GSK3α of less than about 0.050 μM and/or an IC₅₀ for GSK3β of less than about 0.050 μM;
(b) a compound of Formula I: 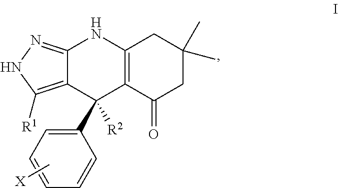
wherein X is hydrogen or halogen;
R¹ is alkyl, unsubstituted or substituted cycloalkyl optionally substituted at one or more positions with halogen, or halogen; and
R² is alkyl; or
(c) a compound selected from the group consisting of N-(3-(1H-1,2,4-triazol-1-yl)propyl)-5-(3-chloro-4-methoxyphenyl)oxazole-4-carboxamide, 3-(5-fluorobenzofuran-3-yl)-4-(5-methyl-5H-[1,3]dioxolo[4,5-f]indol-7-yl)-1H-pyrrole-2,5-dione, 2-hydroxy-3-(5-(morpholinomethyl)pyridin-2-yl)-1H-indole-5-carbonitrile, 3-amino-6-(4-(3-((3-methoxypropyl)amino)propyl)phenyl)-N-(pyridin-3-yl)pyrazine-2-carboxamide, 3-amino-6-(4-((4-methylpiperazin-1-yl)sulfonyl)phenyl)-N-(pyridin-3-yl)pyrazine-2-carboxamide, 5-fluoro-N-(3-fluoro-4-(methylsulfonyl)phenyl)-4-(1-(tetrahydro-2H-pyran-4-yl)-1H-imidazol-5-yl)pyrimidin-2-amine, N-(4-(4-chlorophenyl)pyridin-3-yl)-2-(cyclopropanecarboxamido)isonicotinamide, 2-((4-cyanophenyl)amino)-N-(4-(4-fluorophenyl)pyridin-3-yl)pyrimidine-4-carboxamide, 2-((4-fluorophenyl)amino)-N-(4-(4-fluorophenyl)pyridin-3-yl)pyrimidine-4-carboxamide, 2-((4-(difluoromethoxy)phenyl)amino)-N-(4-(4-fluorophenyl)pyridin-3-yl)pyrimidine-4-carboxamide, (S)-2-(2-(4-fluorophenyl)morpholino)-1-methyl-[4,4′-bipyrimidin]-6(1H)-one, 4-benzyl-2-(naphthalen-1-yl)-1,2,4-thiadiazolidine-3,5-dione, 6-((2-((4-(2,4-dichlorophenyl)-5-(5-methyl-1H-imidazol-2-yl)pyrimidin-2-yl)amino)ethyl)amino)nicotinonitrile, N2-(2-((4-(2,4-dichlorophenyl)-5-(1H-imidazol-1-yl)pyrimidin-2-yl)amino)ethyl)-5-nitropyridine-2,6-diamine, 4-benzyl-2-methyl-1,2,4-thiadiazolidine-3,5-dione, 1-(4-methoxybenzyl)-3-(5-nitrothiazol-2-yl)urea, 3-((3-chloro-4-hydroxyphenyl)amino)-4-(2-nitrophenyl)-1H-pyrrole-2,5-dione, 3-(2,4-dichlorophenyl)-4-(1-methyl-1H-indol-3-yl)-1H-pyrrole-2,5-dione, 3-((6-(3-aminophenyl)-7H-pyrrolo[2,3-d]pyrimidin-4-yl)oxy)phenol, 9-bromo-7,12-dihydropyrido[3′,2′:2,3]azepino[4,5-b]indol-6(5H)-one, 9-bromo-7,12-dihydrobenzo[2,3]azepino[4,5-b]indol-6(5H)-one, 2-methyl-5-(3-(4-(methylsulfinyl)phenyl)benzofuran-5-yl)-1,3,4-oxadiazole, and 3-amino-6-(4-((4-(4-(1-(17-((2-(2,6-dioxopiperidin-3-yl)-1,3-dioxoisoindolin-4-yl)amino)-3,6,9,12,15-pentaoxaheptadecyl)-1H-1,2,3-triazol-4-yl)butyl)piperazin-1-yl)sulfonyl)phenyl)-N-(pyridin-3-yl)pyrazine-2-carboxamide.
